(19) United States
(12) Patent Application Publication
(10) Pub. No.: US 2015/0355199 A1
(43) Pub. Date: Dec. 10, 2015

(54) AGENTS, KITS AND METHODS FOR COMPLEMENT FACTOR H-RELATED PROTEIN 1 DETECTION

(71) Applicant: Roche Diagnostics Operations, Inc., Indianapolis, IN (US)

(72) Inventors: Alfred Engel, Weilheim (DE); Andreas Gallusser, Penzberg (DE); Johann Karl, Peissenberg (DE); Peter Kastner, Benediktbeuern (DE); Wolfgang Obermeier, Peissenberg (DE); Monika Soukupova, Wessobrunn (DE)

(21) Appl. No.: 14/835,821

(22) Filed: Aug. 26, 2015

Related U.S. Application Data
(63) Continuation of application No. PCT/EP2014/053493, filed on Feb. 24, 2014.

(30) Foreign Application Priority Data
Feb. 26, 2013 (EP) 13156823.0

(51) Int. Cl. G01N 33/68; C07K 16/18; G01N 33/543

(52) U.S. Cl. CPC G01N 33/6896; G01N 33/54306; C07K 16/18; G01N 2800/2814; C07K 2317/56; C07K 2317/565; C07K 2317/30

(57) ABSTRACT

The present invention relates to an assay for specific detection of complement factor H-related protein 1 (CFHR1) in a sample from a subject, as well as kits and agents related thereto.

### CROSS REFERENCE TO RELATED APPLICATIONS

This application is a continuation of U.S. International Application No. PCT/EP2014/053493 filed Feb. 24, 2014, which claims priority to European Application No. 13156823.0 filed Feb. 26, 2013, which are hereby incorporated by reference.

### BACKGROUND OF THE INVENTION

Complement factor H, also known as factor H, is a sialic acid containing glycoprotein that plays an integral role in the regulation of the complement-mediated immune system that is involved in microbial defense, immune complex processing, programmed cell death and age-related macula degeneration. Complement factor H is the best characterized member of the complement factor H protein family. The complement factor H family consists of the following members: complement factor H (CFH), complement factor H-related protein 1 (CFHR1), complement factor H-related protein 2 (CFHR2), complement factor H-related protein 3 (CFHR3), complement factor H-related protein 4 with isoforms 4A and 4B (CFHR4A and CFHR4B) and complement factor H-related protein 5 (CFHR5). The complement factor H-related proteins are encoded downstream of the complement factor H gene and share a high concentration of homology with subdomains of complement factor H. Complement factor H related proteins share also functional similarities (Jozsi, M. and Zipfel, P. F., Trend in Immunology 29 (2008) 380-387).

The complement system consists of ˜40 proteins that are present in body fluids or on cell and tissue surfaces and is activated in a cascade-like manner by three major pathways (Walport, M. J. N., Engl. J. Med. 344, 1058-1066). The alternative pathway is activated continuously at a low rate by the spontaneous hydrolysis of the central component C3, the lectin pathway is initiated by mannose binding lectin or ficolins that recognize microbial carbohydrates and the classical pathway is activated by binding of C1q to antigen bound immunoglobulins. Enzymatic steps generate active fragments of complement components and trigger further amplification. The three pathways merge at the concentration of C3, which on activation, is cleaved into C3a and C3b. Complement factor H protects host cells from injury resulting from unrestrained complement activation. Complement factor H regulates complement activation on self cells by possessing both cofactor activity for the Factor I mediated C3b cleavage, and decay accelerating activity against the alternative pathway C3 convertase, C3bBb. Complement factor H protects self cells from complement activation but not bacteria/viruses. Due to the central role that Complement factor H plays in the regulation of complement, there are many clinical implications arising from aberrant CFH activity. Mutations in the Complement factor H gene are associated with severe and diverse diseases including the rare renal disorders hemolytic uremic syndrome (HUS) and membranoproliferative glomerulonephritis (MPGN) also termed dense deposit disease (DDD), membranoproliferative glomuleronephritis type II or dense deposit disease, as well as the more frequent retinal disease age related macular degeneration (AMD). In addition to its complement regulatory activities, complement factor H has multiple physiological activities and 1) acts as an extracellular matrix component, 2) binds to cellular receptors of the integrin type, and 3) interacts with a wide selection of ligands, such as the C-reactive protein, thrombospondin, bone sialoprotein, osteopontin, and heparin.

The Complement factor H protein family comprises the proteins CFH, CFHR1, CFHR2, CFHR3, CFHR4A, CFHR4B and CFHR5.

It would appear that in the prior art is no specific method available for the in vitro detection of complement factor H-related protein 1 (CFHR1) in blood, serum, plasma, liquor samples, or any other body fluid. The inventors of the present invention have now found and could establish a method to determine CFHR1 specifically in a blood, serum, plasma or liquor sample derived from an individual.

It is an object of the present invention to provide a simple and cost efficient procedure of CFHR1 detection in samples, e.g. in order to diagnose diseases and disorders related to CFHR1.

Whole blood, serum or plasma are the most widely used sources of sample in clinical routine. The identification of a marker that would aid in the reliable detection of a certain disease or provide early prognostic information could lead to a method that would greatly aid in the diagnosis and in the management of this disease. It is especially important to improve the early diagnosis of certain diseases, since early intervention may diminish functional disability and improve long-term outcome.

It was the object of the present invention to investigate a method which assesses CFHR1 specifically, i.e. without cross-reactivity to other CFH family members in vitro, preferably in a body fluid sample of a subject.

### SUMMARY OF THE INVENTION

The present invention relates to an assay for specific detecting complement factor H-related protein 1 (CFHR1) in a sample from a subject, as well as kits agents related thereto.

The inventors of the present invention have surprisingly been able to demonstrate a method for the specific detection of complement factor H-related protein 1 (CFHR1), a protein of the complement factor H family member(s), using kits of the present invention.

The method of the present invention is in particular suitable for the in vitro assessment of CFHR1 in a blood, serum, plasma or liquor sample of a subject.

The disclosed methods and kits can overcome several of the problems of the methods available for assessment of CFH family members presently known.

### DESCRIPTION OF THE FIGURES

FIG. 1 shows the protein structure of CFH and CFH related proteins (Józsi M, Zipfel P F “Factor 8 family proteins and human diseases” Trends Immunol. 2008; 29(8):380-7).

FIG. 2 shows the Heavy Chain sequence of monoclonal antibody <CFHR1>M-5.1.5, with CDR regions underlined. The sequences correspond to SEQ ID NO: 15 (DNA), and SEQ ID NO: 16 (Protein).

FIG. 3 shows the Light Chain sequence of monoclonal antibody <CFHR1>M-5.1.5, with CDR regions underlined. The sequences correspond to SEQ ID NO: 17 (DNA), and SEQ ID NO: 18 (Protein).

FIG. 4 shows the amino acid sequences of recombinant CFHR1-CFHR5 and CFHL1-Derivatives.

FIG. 5 shows the Heavy Chain sequence of monoclonal antibody <CFHR1>M-4.1.3, with CDR regions underlined. The sequences correspond to SEQ ID NO: 24 (DNA), and SEQ ID NO: 25 (Protein).

FIG. 6 shows the Light Chain sequence of monoclonal antibody <CFHR1>M-4.1.3, with CDR regions underlined. The sequences correspond to SEQ ID NO: 26 (DNA), and SEQ ID NO: 27 (Protein).

FIG. 7 shows the Heavy Chain sequence of monoclonal antibody <CFHR1>M-4.2.53, with CDR regions underlined. The sequences correspond to SEQ ID NO: 28 (DNA), and SEQ ID NO: 29 (Protein).

FIG. 8 shows the Light Chain sequence of monoclonal antibody <CFHR1>M-4.2.53, with CDR regions underlined. The sequences correspond to SEQ ID NO: 30 (DNA), and SEQ ID NO: 31 (Protein).

FIG. 9 shows the Heavy Chain sequence of monoclonal antibody <CFHR1>M-4.2.74, with CDR regions underlined. The sequences correspond to SEQ ID NO: 32 (DNA), and SEQ ID NO: 33 (Protein).

FIG. 10 shows the Light Chain sequence of monoclonal antibody <CFHR1>M-4.2.74, with CDR regions underlined. The sequences correspond to SEQ ID NO: 34 (DNA), and SEQ ID NO: 35 (Protein).

FIG. 11 shows the Heavy Chain sequence of monoclonal antibody <CFHR1>M-5.3.23, with CDR regions underlined. The sequences correspond to SEQ ID NO: 36 (DNA), and SEQ ID NO: 37 (Protein).

FIG. 12 shows the Light Chain sequence of monoclonal antibody <CFHR1>M-5.3.23, with CDR regions underlined. The sequences correspond to SEQ ID NO: 38 (DNA), and SEQ ID NO: 39 (Protein).

FIG. 13 shows the Heavy Chain sequence of monoclonal antibody MAB<CFH/CFHR1>M-L20/3, with CDR regions underlined. The sequences correspond to SEQ ID NO: 40 (DNA), and SEQ ID NO: 41 (Protein).

FIG. 14 shows the Light Chain sequence of monoclonal antibody MAB<CFH/CFHR1>M-L20/3, with CDR regions underlined. The sequences correspond to SEQ ID NO: 42 (DNA), and SEQ ID NO: 43 (Protein).

FIG. 15 shows a Westen Blot performed using MAK<CFHR1>M-5.1.5 as primary and PAK<M-IgG>S-IgG-POD as secondary antibody. Lane 1 MagicMark XP size standard, lane 4 serum-purified CFH (200 ng/well), lane 5 serum-purified CFH (2000 ng/well). The double-band at 37 kDa corresponds to CFHR1. The smear around 220 kDa is due to the overloaded well in order to achieve better detection of the CFHR1-remainder in the purified CFH.

### DESCRIPTION OF THE SEQUENCES

SEQ ID NO: 1 shows the amino acid sequence of the human complement factor H protein isoform 1 (CFH) as well as isoform 2 (CFHL1); SwissProt database accession number: P08603-1 and P08603-2.
SEQ ID NO: 2 shows the amino acid sequence of the human complement factor H related protein 1 (CFHR1); SwissProt database accession number: Q03591.
SEQ ID NO: 3 shows the amino acid sequence of the human complement factor H related protein 2 (CFHR2); SwissProt database accession number: P36980.
SEQ ID NO: 4 shows the amino acid sequence of the human complement factor H related protein 3 (CFHR3); SwissProt database accession number: Q02985.
SEQ ID NO: 5 shows the amino acid sequence of the human complement factor H related protein 4A (CFHR4A); SwissProt database accession number: C9J7J7.
SEQ ID NO: 6 shows the amino acid sequence of the human complement factor H related protein 4B (CFHR4B); SwissProt database accession number: Q92496.
SEQ ID NO: 7 shows the amino acid sequence of the human complement factor H related protein 5 (CFHR5); SwissProt database accession number: Q9BXR6.
SEQ ID NO: 8 shows the amino acid sequence of CFHR1_{—}1,2-GS-His8, which used as immunogen in the Examples of the present invention.
SEQ ID NO: 9 shows the amino acid sequence of CFHR1_{—}1-5 which used as calibrator material in the Examples of the present invention.
SEQ ID NO: 10 shows the amino acid sequence of CFHR2-GS-Avi-GS-His8.
SEQ ID NO: 11 shows the amino acid sequence of CFHR3-GS-Avi-GS-His8.
SEQ ID NO: 12 shows the amino acid sequence of CFHR4-GS-Avi-GS-His8, wherein CFHR4 Variant B was used.
SEQ ID NO: 13 shows the amino acid sequence of CFHR5-GS-Avi-GS-His8.
SEQ ID NO: 14 shows the amino acid sequence of CFHL1-GS-Avi-GS-His8.
SEQ ID NO: 15 shows the cDNA sequence of the Heavy Chain of monoclonal antibody <CFHR1>M-5.1.5 DNA of the present invention.
SEQ ID NO: 16 shows the amino acid sequence of the Heavy Chain of monoclonal antibody <CFHR1>M-5.1.5 of the present invention.
SEQ ID NO: 17 shows the cDNA sequence of the Light Chain of monoclonal antibody <CFHR1>M-5.1.5 DNA of the present invention.
SEQ ID NO: 18 shows the amino acid sequence of the Light Chain of monoclonal antibody <CFHR1>M-5.1.5 of the present invention.
SEQ ID No. 19 shows the hCDR1 amino acid sequence of the Heavy Chain of monoclonal antibody <CFHR1>M-5.1.5 of the present invention.
SEQ ID No. 20 shows the hCDR2 amino acid sequence of the Heavy Chain of monoclonal antibody <CFHR1>M-5.1.5 of the present invention.
SEQ ID No. 21 shows the hCDR3 amino acid sequence of the Heavy Chain of monoclonal antibody <CFHR1>M-5.1.5 of the present invention.
SEQ ID No. 22 shows the LCDR1 amino acid sequence of the Light Chain of monoclonal antibody <CFHR1>M-5.1.5 of the present invention.
SEQ ID No. 23 shows the LCDR3 amino acid sequence of the Light Chain of monoclonal antibody <CFHR1>M-5.1.5 of the present invention.
SEQ ID NO: 24 shows the cDNA sequence of the Heavy Chain of monoclonal antibody <CFHR1>M-4.1.3 DNA of the present invention.
SEQ ID NO: 25 shows the amino acid sequence of the Heavy Chain of monoclonal antibody <CFHR1>M-4.1.3 of the present invention.
SEQ ID NO: 26 shows the cDNA sequence of the Light Chain of monoclonal antibody <CFHR1>M-4.1.3 DNA of the present invention.
SEQ ID NO: 27 shows the amino acid sequence of the Light Chain of monoclonal antibody <CFHR1>M-4.1.3 of the present invention.
SEQ ID NO: 28 shows the cDNA sequence of the Heavy Chain of monoclonal antibody <CFHR1>M-4.2.53 DNA of the present invention.
SEQ ID NO: 29 shows the amino acid sequence of the Heavy Chain of monoclonal antibody <CFHR1>M-4.2.53 of the present invention.
SEQ ID NO: 30 shows the cDNA sequence of the Light Chain of monoclonal antibody <CFHR1>M-4.2.53 DNA of the present invention.
SEQ ID NO: 31 shows the amino acid sequence of the Light Chain of monoclonal antibody <CFHR1>M-4.2.53 of the present invention.
SEQ ID NO: 32 shows the cDNA sequence of the Heavy Chain of monoclonal antibody <CFHR1>M-4.2.74 DNA of the present invention.
SEQ ID NO: 33 shows the amino acid sequence of the Heavy Chain of monoclonal antibody <CFHR1>M-4.2.74 of the present invention.
SEQ ID NO: 34 shows the cDNA sequence of the Light Chain of monoclonal antibody <CFHR1>M-4.2.74 DNA of the present invention.
SEQ ID NO: 35 shows the amino acid sequence of the Light Chain of monoclonal antibody <CFHR1>M-4.2.74 of the present invention.
SEQ ID NO: 36 shows the cDNA sequence of the Heavy Chain of monoclonal antibody <CFHR1>M-5.3.23 DNA of the present invention.
SEQ ID NO: 37 shows the amino acid sequence of the Heavy Chain of monoclonal antibody <CFHR1>M-5.3.23 of the present invention.
SEQ ID NO: 38 shows the cDNA sequence of the Light Chain of monoclonal antibody <CFHR1>M-5.3.23 DNA of the present invention.
SEQ ID NO: 39 shows the amino acid sequence of the Light Chain of monoclonal antibody <CFHR1>M-5.3.23 of the present invention.
SEQ ID NO: 40 shows the cDNA sequence of the Heavy Chain of monoclonal antibody MAB<CFH/CFHR1>M-L20/3 DNA of the kit of the present invention.
SEQ ID NO: 41 shows the amino acid sequence of the Heavy Chain of monoclonal antibody MAB<CFH/CFHR1>M-L20/3 of the kit of the present invention.
SEQ ID NO: 42 shows the cDNA sequence of the Light Chain of MAB<CFH/CFHR1>M-L20/3 DNA of the kit of the present invention.
SEQ ID NO: 43 shows the amino acid sequence of the Light Chain of MAB<CFH/CFHR1>M-L20/3 of the kit of the present invention.

### DETAILED DESCRIPTION OF THE INVENTION

In one embodiment, the present invention relates to a kit comprising
a) a first agent capable of binding complement factor H R1 (CFHR1) protein, and
b) a second agent capable of binding complement factor H R1 (CFHR1) protein,
   wherein the first agent and the second agent bind to different and non-overlapping epitopes,
   and wherein the first agent and the second agent do not both crossreact with CFH,
   and wherein the first agent and the second agent do not both crossreact with CFHR2,
   and wherein the first agent and the second agent do not both crossreact with CFHR3,
   and wherein the first agent and the second agent do not both crossreact with CFHR4,
   and wherein the first agent and the second agent do not both crossreact with CFHR5,
   and wherein one agent is labeled with a detectable label,
   and wherein the other agent is capable of immobilizing on a solid phase.

In a preferred embodiment, the non-overlapping epitopes are epitopes located in different domains of CFHR1.

It was surprisingly found that such a kit allows for specific detection of CFHR1 in samples from a subject, as shown in Examples 7, 8, 9 and 10. In particular, a kit comprising labeled monoclonal antibody MAB<CFHR1>M-5.1.5 and biotin-labeled antibody MAB<CFH/CFHR1>M-L20/3 was successfully used. As described in the Examples, biotin-labeled antibody MAB<CFH/CFHR1>M-L20/3 is capable of immobilizing on a solid phase via binding to magnetic particles coated with streptavidin. Also, MAB<CFHR1>M-5.1.5 is ruthenylated and can be detected by electrochemiluminescence. Also, the two antibodies bind to different and non-overlapping epitopes, as an immunogen corresponding to amino acids 1-143 of CHFR1 (=SCR (short consensus repeat) domains 1-2 of CHFR1) was used for generating MAB<CFHR1>M-5.1.5, whereas MAB<CFH/CFHR1>M-L20/3 binds to an epitope within the more C-terminal part of CFHR1, i.e. within SCR domains 3-4-5 of CHFR1.

The first agent and the second agent are understood as polypeptides or polypeptide complexes.

Therefore, in a preferred embodiment of the kit of the invention, the first agent binds to an epitope within amino acids 144-330 of SEQ ID No. 2.

Therefore, in another preferred embodiment of the kit of the invention, the second agent binds to an epitope within amino acids 1-143 of SEQ ID No. 2.

As shown in Table 1, MAB<CFHR1>M-5.1.5 does not crossreact with CFH, but shows crossreaction with CFHR2. Also, Table 1 shows that MAB<CFH/CFHR1>M-L20/3 crossreacts with CFH and CFHR5, but not with CFHR2.

Lack of cross-reaction with CFHR2 and/or CFH and CFHR5, respectively, are critically important, as these members of the CFH protein family show the greatest structural similarity with CFHR1.

Therefore, in another preferred embodiment of the kit of the invention the first agent does not crossreact with CFHR2.

In another preferred embodiment of the kit of the invention the second agent does not crossreact with CFH.

In another preferred embodiment of the kit of the invention the second agent does not crossreact with CFHR5.

In another preferred embodiment of the kit of the invention the second agent does not crossreact with CFH and CFHR5.

In a preferred embodiment of the kit, the first agent crossreacts with CFH and CFHR5, but does not crossreact with CFHR2, CFHR3, and CFHR4.

In another preferred embodiment of the kit, the second agent crossreacts with CFHR2, but does not crossreact with CFH, CFHR3, CFHR4 and CFHR5.

According to the present invention “crossreact” means, that the binding strength to a protein in question distinct from the target protein against which an agent, in particular an antibody, is directed, is at least 0.2%, preferably at least 0.1% of the binding strength measured with the target protein. Binding strength can in particular be measured by applying the affinity test of Examples of the present invention using a BiaCore. As the skilled artisan knows, the binding strengths, if given as Kd is the better/higher, the lower the Kd.

CFH is comparably abundant in certain body fluids, with a concentration of about 10 times of the concentration of CFHR1.

The two agents used in the invention may exhibit cross-reactivity to other member of the CFH family. However, the two agents used in the invention, do not cross-react with the same member of the CFH family, i.e. at most one of them cross-reacts with CFH, CFHR2, CFHR3, CFHR4 and CFHR5, respectively.

Therefore, in a preferred embodiment of the kit of the invention the first agent may crossreact with CFH, but does not crossreact with more than one other CFH family member.

Therefore, in a another more preferred embodiment of the kit of the invention the first agent may crossreact with CFHR5, but does not crossreact with more than one other CFH family member.

Therefore, in a further even more preferred embodiment of the kit of the invention the first agent may crossreact with CFHR5 and CFH, but does not crossreact with the other CFH family members.

Therefore, in another preferred embodiment of the kit of the invention the second agent may crossreact with CFHR2, but does not crossreact with the other CFH family members.

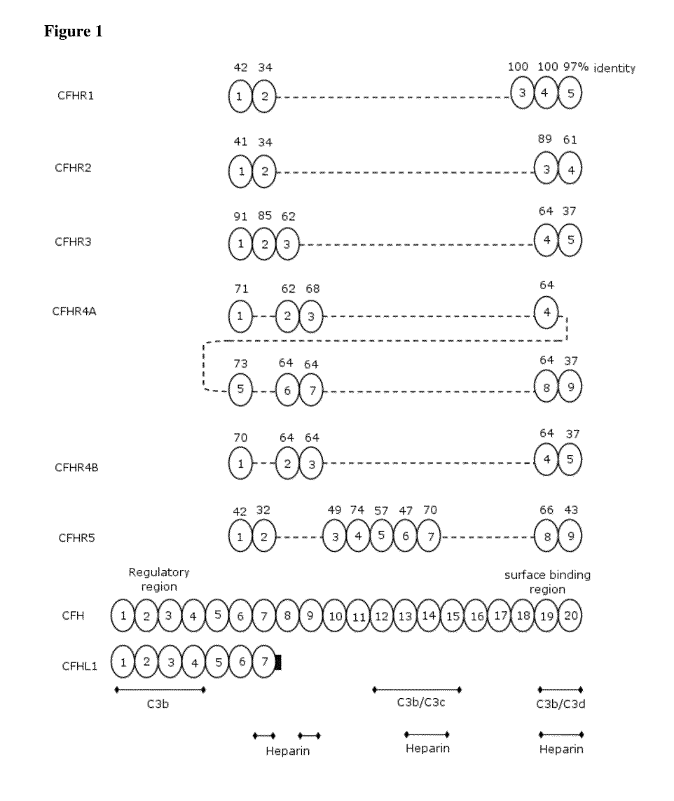

“CFHR4” according to the present application encompasses the naturally occurring variants CFHR4A and CFHR4B. As can be seen from FIG. 1 of the present application, two isoforms of CFHR4 exist, namely CFHR4A, as disclosed in SEQ ID No. 5, and CFHR4B, as disclosed in SEQ ID No. 6. CFHR4A and CFHR4B have an identical N-terminal sequence, but CFHR4B has a shorter C-terminal portion. The gene transcript coding for CFHR4 is alternatively spliced and different variants of CFHR4 are expressed. There are variants coding for CFHR4 polypeptides with a length of 331 amino acids as well as for a 577 amino acid variant. The latter is termed “CFHR4A”, whereas a short isoform is termed “CFHR4B”. Both variants are expressed in human liver, yet only the short isoform was cloned and expressed for use in the present invention. Homology analysis of both variants showed that SCR domain 1 is identical in both variants. CFHR4A SCR2-5 is almost identical to CFHR4B SCR2-4 with only a few amino acids difference. Further CFHR4B SCR6-9 share 100% identity with CFHR4A SCR2-5 (Joszi, Richter, Löschmann et al., European Journal of Human Genetics, 2005, 13, 321-329). Therefore CFHR4A was not cloned and expressed or used in the cross-reactivity assays since the domain composition is represented by CFHR4B already. In the Experiments, CFHR4B was therefore used in order to determine crossreactivity to CFHR4. Therefore, the experimental results obtained for CFHR4B also apply for variant CFHR4A, and therefore to CFHR4 in general.

In an even more preferred embodiment, the first agent is an antibody, in particular the antibody MAB<CFH/CFHR1>M-L20/3. In particular, the heavy chain of MAB<CFH/CFHR1>M-L20/3 has a sequence of SEQ ID No. 41, and the light chain has a sequence of SEQ ID No. 43. In a further preferred embodiment, an antibody comprising the CDR sequences of MAB<CFH/CFHR1>M-L20/3 is used.

In an even more preferred embodiment, the first agent is an antibody, in particular the antibody MAB<CFHR1>M-5.1.5. In particular, the heavy chain has a sequence of SEQ ID No. 16. In a further preferred embodiment, the light chain has a sequence of SEQ ID No. 18. In a further preferred embodiment, an antibody comprising the CDR sequences of MAB<CFHR1>M-5.1.5 according to SEQ ID No. 19, SEQ ID No. 20, SEQ ID No. 21, SEQ ID No. 22, and SEQ ID No. 23 and the amino acid sequence ITS is used.

The term “detectable label” as used herein refers to any substance that is capable of producing a signal via direct or indirect detection. The detectable label thus may be detected directly or indirectly. For direct detection label suitable for use in the present invention can be selected from any known detectable marker groups, like chromogens, fluorescent groups, chemiluminescent groups (e.g. acridinium esters or dioxetanes), electrochemiluminescent compounds, catalysts, enzymes, enzymatic substrates, dyes, fluorescent dyes (e.g. fluorescein, coumarin, rhodamine, oxazine, resorufin, cyanine and derivatives thereof), colloidal metallic and nonmetallic particles, and organic polymer latex particles. Other examples of detectable labels are luminescent metal complexes, such as ruthenium or europium complexes, e.g. as used for ECLIA, enzymes, e.g. as used for ELISA, and radioisotopes; e.g. as used for RIA.

Indirect detection systems comprise, for example, that the detection reagent, e.g. the detection antibody, is labeled with a first partner of a bioaffine binding pair. Examples of suitable binding pairs are hapten or antigen/antibody, biotin or biotin analogues such as aminobiotin, iminobiotin or desthiobiotin/avidin or streptavidin, sugar/lectin, nucleic acid or nucleic acid analogue/complementary nucleic acid, and receptor/ligand, e.g. steroid hormone receptor/steroid hormone. Preferred first binding pair members comprise hapten, antigen and hormone. Especially preferred are haptens like digoxin and biotin and analogues thereof. The second partner of such binding pair, e.g. an antibody, streptavidin, etc., usually is labeled to allow for direct detection, e.g. by the detectable labels as mentioned above.

In a preferred embodiment, the kits of the present invention further comprise auxiliary reagents for performing the measurement.

In one preferred embodiment, the kits of the present invention further comprise a chip on which an agent can be immobilized.

The first agent and the second agent of the kits of the present invention bind to different and non-overlapping epitopes. Such epitopes may be linear or conformational. The first agent and the second agent can bind to their respective epitopes on CFHR1 without interfering with the binding of the respective other agent.

The present invention allows for the first time the specific detection of CFHR1 in a sample of a subject. Also, this allows for the first time the diagnosis of CFHR1-related diseases and disorders, such as schizophrenia.

In one embodiment the present invention relates to a method for specifically measuring complement factor H related protein 1 (CFHR1) in a sample comprising the steps of contacting the sample with a first agent capable of binding complement factor H R1 (CFHR1) protein, and a second agent capable of binding complement factor H R1 (CFHR1) protein, thereby forming a complex between said first agent, CFHR1 and said second agent and b) measuring the complex formed in (a), wherein the first agent and the second agent both bind to CFHR1 and do not both cross-react with the same CFH-family member other than CFHR1.

In a further embodiment, the present invention relates to an in vitro assay for detecting complement factor H R1 (CFHR1) protein in a sample obtained from a subject, comprising
a) contacting the sample with the agents of any of the kits of the present invention,
b) immobilizing the formed complexes to a solid phase, and
c) detecting CFHR1,
   wherein step b) may be performed before step a), after step a) or simultaneously with step a).

In a preferred embodiment, one agent capable of binding to CFHR1 may be immobilized to a solid support prior to contacting the precoated solid phase with the sample, which can be incubated with the solid phase simultaneously or sequentially with the other agent(s) of any of the kits of the present invention.

In a further embodiment one agent capable of binding to CFHR1 may be immobilized to a solid support while contacting the sample with the other agent(s) of any of the kits of the present invention. In this embodiment, immobilizing the formed complexes to a solid phase will occur simultaneously with step a).

In a preferred embodiment, the agent with less cross-reactivity to CFH, CFHR2, CFHR3, CFHR4 and/or CFHR5 is contacted with the sample according to step a) before contacting the other agent with the sample.

In a preferred embodiment, the amount and/or concentration of CFHR1 is determined in step c).

When performing a method of the present invention, CFHR1 and the first and second agents as described above form a complex, wherein each the first and second agent binds to CFHR1. The complex formation may be through covalent or non-covalent binding of the first and second agent to CFHR1, preferably through non-covalent binding. Therefore, a “formed complex” according to the invention is understood as complex comprising CFHR1, a first and a second agent as defined above, wherein binding between the three molecules may be covalently or non-covalently.

It is possible to immobilize the agent which is capable of immobilizing on a solid phase, to a solid phase prior to step a). Upon contacting the sample with the agents according to the present invention, the immobilized formed complex will form at the same time.

Alternatively, the formed complexes are immobilized after complex formation, as described in Example 7. In Example 7, the formed complexes are immobilized via non-covalent binding to magnetic particles which are then immobilized on a surface using electrodes. In this embodiment, step b) is performed after step a).

In general, immobilization may be performed directly or indirectly, and by covalent or non-covalent means.

In a preferred embodiment, immobilization occurs on magnetic particles. An agent of the invention can be bound to such magnetic particles via covalent or non-covalent binding. In Example 7, binding occurs via biotin-streptavidin binding. The magnetic particles are coated with streptavidin, whereas an antibody is biotinylated.

In a preferred embodiment, a bioaffine binding pair is used for immobilization. Examples of suitable binding pairs are hapten or antigen/antibody, biotin or biotin analogues such as aminobiotin, iminobiotin or desthiobiotin/avidin or streptavidin, sugar/lectin, nucleic acid or nucleic acid analogue/complementary nucleic acid, and receptor/ligand, e.g. steroid hormone receptor/steroid hormone. Preferred first binding pair members comprise hapten, antigen and hormone. Especially preferred are haptens like digoxin and biotin and analogues thereof. The second partner of such binding pair, e.g. an antibody, streptavidin, etc., is usually bound to a solid phase, or is covalently attached to such solid phase, e.g. to magnetic beads.

In some embodiments, the solid phase is a test strip, a chip, in particular a microarray or nanoarray chip, a microtiter-plate or a microparticle.

It was found that an in vitro determination of the concentration of CFHR1 in a sample allows the prediction of a clinical benefit from the treatment with Glycine Reuptake Inhibitors (GRI) for patients with neurodevelopmental, neurological or neuropsychiatric disorders.

Glycine Reuptake Inhibitors (GRI) are a novel class of compounds that are thought to enhance NMDA receptor (NMDA-R) mediated transmission by elevating extracellular concentrations of glycine. Evidence from studies in healthy individuals, psychotic patients and animals as well as from genetic analysis has accumulated over the past 15 years of the involvement of NMDA receptor (NMDA-R) hypofunction in the pathophysiology of neurodevelopmental, neurological or neuropsychiatric disorders. As glycine is an obligatory co-agonist at the NMDA-R complex, one strategy to enhance NMDA-R mediated neurotransmission is to elevate extracellular concentrations of glycine in the local microenvironment of NMDA receptors. Glycine elevation can be achieved by inhibition of GRI, which is responsible for glycine removal from the synaptic cleft. Possible advantages over the existing neurological and neuropsychiatric therapies include the potential of glycine reuptake inhibitors in having good efficacy, as well as an improved tolerability profile for the treatment of negative and positive symptoms in schizophrenia (positive and negative symptoms), bipolar disorders, substance dependence (alcohol, cocaine), autism or obsessive compulsive disorders (OCD). It is known that glycine reuptake inhibitors may be used for the treatment of neurodevelopmental, neurological or neuropsychiatric disorders, such as schizophrenia.

Schizophrenia is a severe mental disorder typically appearing in late adolescence or early adulthood with a word-wide prevalence of approximately 1% of the adult population which has enormous social and economic impact. The criteria of the Association of European Psychiatrists (ICD) and the American Psychiatric Association (DSM) for the diagnosis of schizophrenia require that two or more characteristic symptoms be present: delusions, hallucinations, disorganized speech, grossly disorganized or catatonic behavior, or negative symptoms (alogia, affective flattening, lack of motivation, anhedonia), and that other requirements, such as excluding affective disorders, and the presence of impaired function, be present. As a group, people with schizophrenia have functional impairments that may begin in childhood, continue throughout adult life and make most patients unable to maintain normal employment or otherwise have normal social function. They also have a shortened lifespan compared to the general population, and suffer from an increased prevalence of a wide variety of other neuropsychiatric syndromes, including serious, substance abuse, obsessive-compulsive symptoms and abnormal involuntary movements prior to antipsychotic treatment. Schizophrenia is also associated with a wide range of cognitive impairments, the severity of which limits their function, even when psychotic symptoms are well controlled.

Other indications associated with glutamatergic transmission are bipolar disorders, substance dependence (alcohol, cocaine), autism and obsessive compulsive disorders (OCD).

Therefore, the present invention also relates to an in vitro method of predicting a response for patients, having neurodevelopmental, neurological or neuropsychiatric disorders, if treated with a glycine reuptake inhibitor (GRI), comprising the steps:
i) determining the protein concentration of CFHR1 in a sample of a patient by performing an assay of the invention,
ii) comparing the protein concentration determined in step i) to a cut-off value for CFHR1 in patients, having neurodevelopmental, neurological or neuropsychiatric disorders,
iii) wherein a protein concentration CFHR1 in the sample of the patient having neurodevelopmental, neurological or neuropsychiatric disorders above the cut-off value is indicative for a patient who will derive clinical benefit from treatment GRI, and
iv) selecting a GRI treatment for patients having neurodevelopmental, neurological or neuropsychiatric disorders.

In a further preferred embodiment, the neurodevelopmental, neurological or neuropsychiatric disorders include negative or positive symptoms of schizophrenia, bipolar disorder, substance dependence, autism and compulsive disorders, in particular negative or positive symptoms of schizophrenia.

In a further preferred embodiment, the patient is affected with schizoaffective disorder.

Therefore, in one embodiment, the present invention relates to the use of a kit of the invention for determining the amount and/or concentration of CFHR1 in a sample obtained from a subject.

Moreover, it was found that responders and non-responders for a treatment with GRI could reliably be identified by determining CFHR1 concentrations in a body fluid.

Therefore, in a further embodiment, the present invention relates to the use of a kit of the invention for the prediction of the clinical benefit for patients who are treated with a glycine reuptake inhibitor.

Therefore, in a further embodiment, the present invention relates to the use of a kit of the invention for the prediction of the clinical benefit for patients, having neurodevelopmental, neurological or neuropsychiatric disorders, if treated with a glycine reuptake inhibitor (GRI).

For example, the cut-off value for CFHR1 baseline serum values may be calculated as 10 ug/ml and patients with CFHR1 values below or equal 10 ug/ml may be stratified as “CFHR1-low”, while patients with complement factor H serum values above 10 ug/ml may be stratified as “CFHR1-high”. While the placebo treated patients does not show much difference in both subgroups, the patients treated with 10 mg and 30 mg GRI show a stronger response in the CFHR1-high group.

In a further embodiment, the present invention relates to method of diagnosing a CFHR1-related disease or disorder, comprising the steps of:
a) contacting a sample obtained from a subject with the agents of any of the kits of any of the kits of the present invention,
b) immobilizing an agent to a solid phase; and
c) determining the amount of CFHR1,
   wherein step b) may be performed before step a), after step a) or simultaneously with step a), and wherein an altered amount of CFHR1 relative to a control is indicative of a CFHR1-related disease or disorder.

In a preferred embodiment, the agent with less cross-reactivity to CFH is contacted with the sample according to step a) before contacting the other agent with the sample.

The methods and kits of the invention are in particular suitable for detecting CFHR1 and for determining the amount and/or concentration of CFHR1 in liquid samples from a subject. Preferred liquids from a subject are blood, serum, liquor and plasma. Therefore, in a preferred embodiment of the present invention, in methods of the present invention, said sample is blood, serum, liquor, plasma or another body fluid. In one embodiment the sample is selected from serum or plasma.

The present invention allows for the first time to diagnose the presence or absence of schizophrenia, and/or the severity of schizophrenia in a subject, by specifically detecting CFHR1 in a sample from subject.

Also, the present invention allows for the first time to reliably determine the amount and/or concentration of CFHR1 protein in a sample of a subject, in particular wherein the concentration above or below a cut-off value, is indicative for the presence and/or severity of a disease.

In a further preferred embodiment of the present invention, the CFHR1-related disease or disorder is selected from schizophrenia, rare renal disorders hemolytic uremic syndrome (HUS) or atypical HUS (aHUS), and membranoproliferative glomerulonephritis (MPGN) also termed dense deposit disease (DDD), membranoproliferative glomuleronephritis type II or dense deposit disease, and retinal disease age related macular degeneration (AMD), more preferably, the CFHR1-related disease or disorder is schizophrenia.

The absence of CFHR1 in plasma has opposite effects on the progression of the following disorders: In aHUS, the deletion seems to represent a risk factor, whereas in AMD it is described as having a protective effect (P. Zipfel and Skerka, Nature Reviews Immunology, 2009 9 (10): 729-740). Deletion of complement factor H-related 1 (CFHR1) increases the risk of aHUS (Zipfel P. et al., 2007, PLoS Genet 3(3): e41). Deletion of CFHR1 is associated with lower risk of age-related macular degeneration (A. Hughes, Nature genetics, 2006, 38, 1173-1177).

Therefore, in one embodiment, in case of aHUS, a concentration above a cut-off value, is indicative for the presence and/or severity of this disease.

Therefore, in another embodiment, in case of AMD, a concentration below a cut-off value, is indicative for the presence and/or severity of this disease.

A suitable cut-off value may be determined as known by persons skilled in the art and as described below in further detail.

Also, in a further preferred embodiment of the present invention, the amount and/or concentration of CFHR1 protein above or below a reference amount and/or concentration and/or cut-off value is indicative for the presence and/or severity of a disease.

Therefore, in one embodiment, the kits of the invention may be used for determining the amount of CFHR1 in a sample obtained from a subject, and/or for the in vitro diagnosis of a CFHR1-related disease or disorder.

The readout of the assay depends on the detectable label used. In the Examples, a ruthenylated antibody was used. Such antibody was detected by measuring electrochemiluminescence. Alternatively, an antibody linked to any other appropriate label, e.g. linked to an enzyme may be used. Such enzyme can then be used for generating a detectable substance.

In an embodiment in a method according to the present invention CFHR1 is measured in an immunoassay procedure.

Immunoassays are well known to the skilled artisan. Methods for carrying out such assays as well as practical applications and procedures are summarized in related textbooks. Examples of related textbooks are Tijssen, P., Preparation of enzyme-antibody or other enzyme-macromolecule conjugates, In: Practice and theory of enzyme immunoassays, pp. 221-278, Burdon, R. H. and v. Knippenberg, P. H. (eds.), Elsevier, Amsterdam (1990), and various volumes of Methods in Enzymology, Colowick, S. P., and Caplan, N. O. (eds.), Academic Press), dealing with immunological detection methods, especially volumes 70, 73, 74, 84, 92 and 121.

In a preferred embodiment, CFHR1 is detected in a sandwich assay.

In a preferred embodiment CFHR1 is detected in an enzyme-linked immunoassay (ELISA). CFHR1 is detected in a further preferred embodiment in an (electro-) chemiluminescence immunoassay (ECLIA). CFHR1 is detected in a further embodiment in a radioimmunoassay (RIA). Further preferred assays are sandwich fluorescence immunoassay (FIA), Microparticle capture enzyme immunoassay (MEIA), Solid-phase fluorescence immunoassays (SPFIA), Particle concentration fluorescence immunoassay (PCFIA), Nephelometric and Turbidimetric assay with and without latex particle enhancement (LPIA). Also, the assay may be in the form of test strips.

In a preferred embodiment, a sandwich immunoassay is used in order to determine CFHR1 in a sample. As shown in the Examples, such sandwich immunoassay specifically detects CFHR1 in a sample.

In a sandwich assay, a first agent is used to capture CFHR1 on the one side and a second agent, which is labeled to be directly or indirectly detectable, is used on the other side. The agents used in a sandwich-type assay format may be antibodies binding CFHR1. The agents of the kits of the invention bind to non-overlapping epitopes.

In one embodiment, the kits of the present invention are used for a qualitative (CFHR1 present or absent) or quantitative (amount of CFHR1 is determined) or semi-quantitative (relative amounts, in particular above or below a cut-off value are given) immunoassay.

In a preferred embodiment CFHR1 is detected in an electrochemical or electrochemiluminescence immunoassay (=ECLIA). In an electrochemical or electrochemiluminescent assay a bound analyte molecule is detected by a label linked to a detecting agent (target molecule). An electrode electrochemically initiates luminescence of a chemical label linked to a detecting agent. Light emitted by the label is measured by a photodetector and indicates the presence or quantity of bound analyte molecule/target molecule complexes. ECLA methods are described, for example, in U.S. Pat. Nos. 5,543,112; 5,935,779; and 6,316,607. Signal modulation can be maximized for different analyte molecule concentrations for precise and sensitive measurements.

Moreover, it could be shown in Examples 9 and 10, that the assay of the invention does not show any significant crossreactivity to other members of the CFH family; i.e. the cross-reactivity to any member of the CFH family other than CFHR1 has been found to be far less than 0.2%, in particular less than 0.1%. Thus, also small amounts of CFHR1 can be detected specifically and reliably, even in the presence of other CFH family members.

In a preferred embodiment, the detection range of CFHR1 protein is from about 0.02 to about 50 μg/ml, more preferred from about 0.05 to about 35 μg/ml.

In the Examples of the present invention, the antibody MAB<CFH/CFHR1>M-L20/3 known in the art and MAB<CFHR1>M-5.1.5 of the present invention were used successfully.

Therefore, in a preferred embodiment, the first agent and/or second agent is/are antibodies, in particular monoclonal antibodies.

In a more preferred embodiment, the first agent is MAB<CFH/CFHR1>M-L20/3, which is labeled with a detectable label or capable of immobilizing on a solid phase. In a more preferred embodiment, the first agent is MAB<CFH/CFHR1>M-L20/3, which is capable of immobilizing on a solid phase. In an even more preferred embodiment, the first agent is MAB<CFH/CFHR1>M-L20/3, which antibody is biotinylated.

In a further preferred embodiment, the first agent is capable of immobilizing on a solid phase.

In another more preferred embodiment, the second agent is MAB<CFHR1>M-5.1.5, which is labeled with a detectable label or capable of immobilizing on a solid phase. In a more preferred embodiment, the second agent is MAB<CFHR1>M-5.1.5, which is labeled with a detectable label. In an even more preferred embodiment, the second agent is MAB<CFHR1>M-5.1.5, which is ruthenylated.

“MAB<CFHR1>M-5.1.5” is understood as monoclonal antibody wherein the heavy chain has a sequence according to SEQ ID No. 16, and wherein the light chain has a sequence according to SEQ ID No. 18. MAB<CFHR1>M-5.1.5 binds to CFHR1.

“MAB<CFH/CFHR1>M-L20/3” is understood as monoclonal antibody wherein the heavy chain has a sequence according to SEQ ID No. 41, and wherein the light chain has a sequence according to SEQ ID No. 43 or as “anti-Complement Factor H, Clone: L20/3” available from Thermo Scientific as catalogue number GAU 020-03-02.

For the assay of the invention, at least one agent is labeled with a detectable label, and at least one agent is capable of immobilizing on a solid phase. In a preferred embodiment, one agent is labeled with a detectable label, and one agent is capable of immobilizing on a solid phase.

Therefore, in one embodiment of the methods of the present invention, the first agent is capable of immobilizing on a solid phase and the second agent is labeled with a detectable label. Therefore, in one further embodiment of the methods of the present invention the first agent is labeled with a detectable label and the second agent is immobilized on a solid phase.

In a preferred embodiment, the first agent is capable of immobilizing on a solid phase, more preferably MAB<CFH/CFHR1>M-L20/3 is capable of immobilizing on a solid phase. In one embodiment, MAB<CFH/CFHR1>M-L20/3 is biotinylated.

In another preferred embodiment, the second agent is labeled with a detectable label, more preferably MAB<CFHR1>M-5.1.5 is labeled with a detectable label. In one embodiment, MAB<CFHR1>M-5.1.5 is ruthenylated.

It is preferred to standardize the assay using a calibrator. In a more preferred embodiment, such calibrator protein is produced recombinantly, in particular in HEK cells.

As shown in Example 4, MAB<CFHR1>M-5.1.5 was identified as a monoclonal antibody exhibiting high affinity to CFHR1 and showing only low crossreactivity to other members of the CFH family. Notably, only crossreactivity to CFHR2 was detectable (see Table 2). Moreover, MAB<CFHR1>M-5.1.5 surprisingly allowed for the first time a reliable and sensitive detection of CFHR1 in a sample of a subject (see Example 9). The sequence of the heavy chain of MAB<CFHR1>M-5.1.5 is SEQ ID No. 16. The sequence of the light chain of MAB<CFHR1>M-5.1.5 is SEQ ID No. 18.

Therefore, the invention also relates to an agent, in particular antibody, capable of binding of CFHR1, which antibody is monoclonal antibody MAB<CFHR1>M-5.1.5, wherein the heavy chain has a sequence of SEQ ID No. 16, and wherein the light chain has a sequence of SEQ ID No. 18.

Moreover, further antibodies were identified according to Examples 3 and 4, which show binding to CFHR1 and which may also be used in the kits and methods of the present invention, as shown in Example 9.

Therefore, in a further embodiment, the present invention relates to
(i) monoclonal antibody MAB<CFHR1>M-4.1.3, wherein the heavy chain has a sequence of SEQ ID No. 25, and wherein the light chain has a sequence of SEQ ID No. 27, or
(ii) monoclonal antibody MAB<CFHR1>M-4.2.53, wherein the heavy chain has a sequence of SEQ ID No. 29, and wherein the light chain has a sequence of SEQ ID No. 31, or
(iii) monoclonal antibody MAB<CFHR1>M-4.2.74, wherein the heavy chain has a sequence of SEQ ID No. 33, and wherein the light chain has a sequence of SEQ ID No. 35, or
(iv) monoclonal antibody MAB<CFHR1>M-5.3.23, wherein the heavy chain has a sequence of SEQ ID No. 37, and wherein the light chain has a sequence of SEQ ID No. 39.

In a further embodiment, the present invention relates an agent, in particular an antibody, capable of binding of CFHR1 comprising the CDR sequences of MAB<CFHR1>M-5.1.5 according to SEQ ID No. 19, SEQ ID No. 20, SEQ ID No. 21, SEQ ID No. 22, and SEQ ID No. 23 and the amino acid sequence ITS. The CDR1 sequence of the heavy chain of MAB<CFHR1>M-5.1.5 has the sequence according to SEQ ID No. 19. The CDR2 sequence of the heavy chain of MAB<CFHR1>M-5.1.5 has the sequence according to SEQ ID No. 20. The CDR3 sequence of the heavy chain of MAB<CFHR1>M-5.1.5 has the sequence according to SEQ ID No. 21. The CDR1 sequence of the light chain of MAB<CFHR1>M-5.1.5 has the sequence according to SEQ ID No. 22. The CDR2 sequence of the light chain of MAB<CFHR1>M-5.1.5 has the amino acid sequence ITS. The CDR3 sequence of the light chain of MAB<CFHR1>M-5.1.5 has the sequence according to SEQ ID No. 23.

In a further embodiment, the present invention relates to an agent, in particular antibody, capable of binding CFHR1 comprising the CDR3 sequences of the heavy and light chain of MAB<CFHR1>M-5.1.5 according to SEQ ID No. 21 and 23.

In a yet further embodiment, the present invention relates to an antibody comprising the CDR sequences of MAB<CFHR1>M-4.1.3, MAB<CFHR1>M-4.2.53, MAB<CFHR1>M-4.2.74, or MAB<CFHR1>M-5.3.23 and/or comprising the CDR3 sequences of MAB<CFHR1>M-4.1.3, MAB<CFHR1>M-4.2.53, MAB<CFHR1>M-4.2.74, or MAB<CFHR1>M-5.3.23.

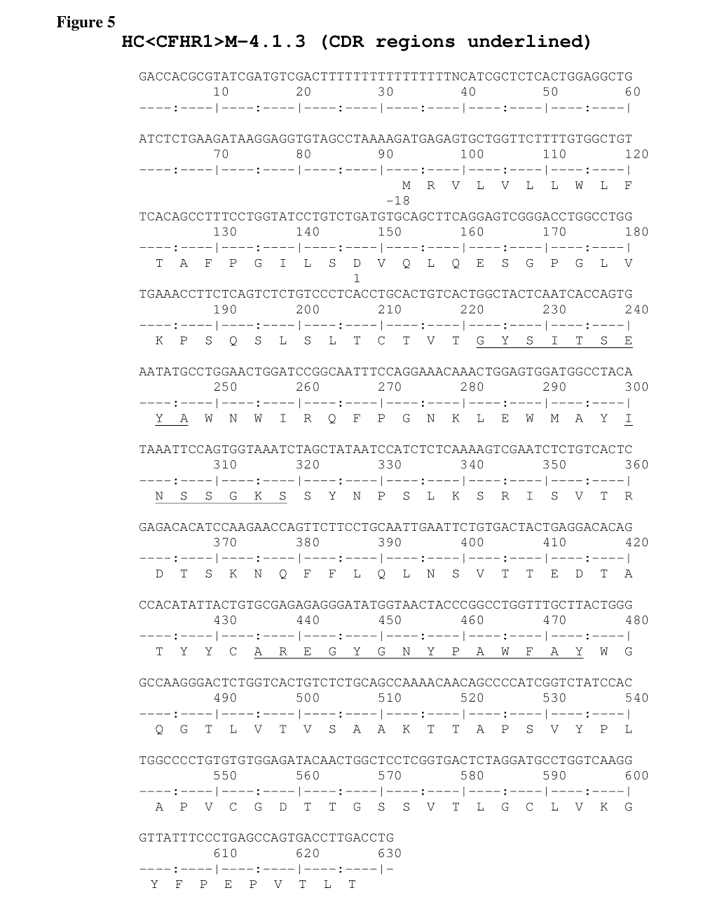

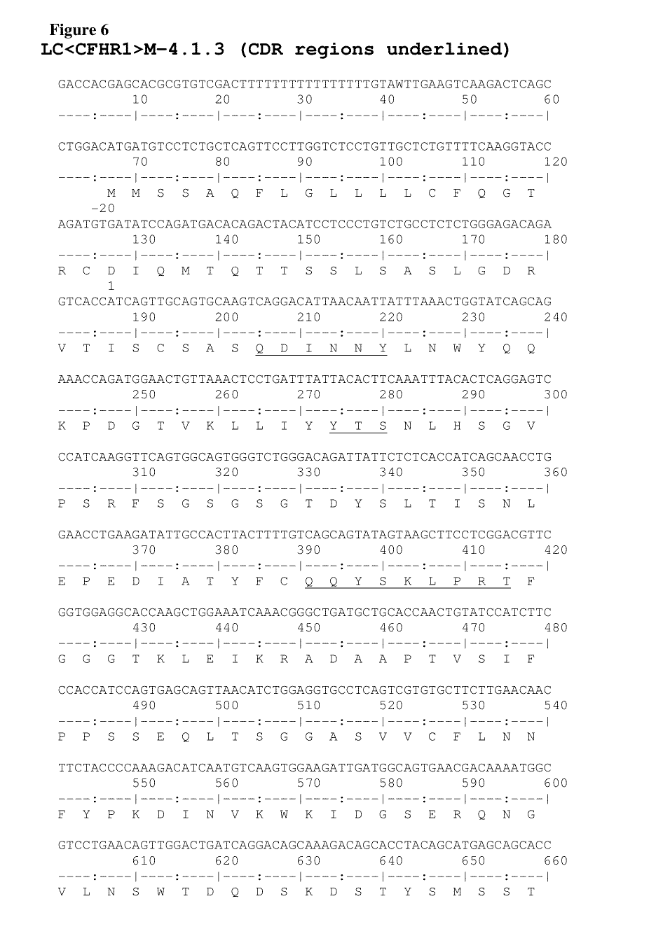

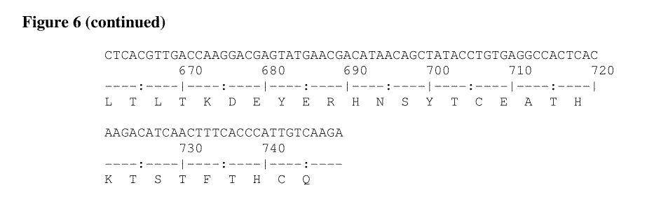

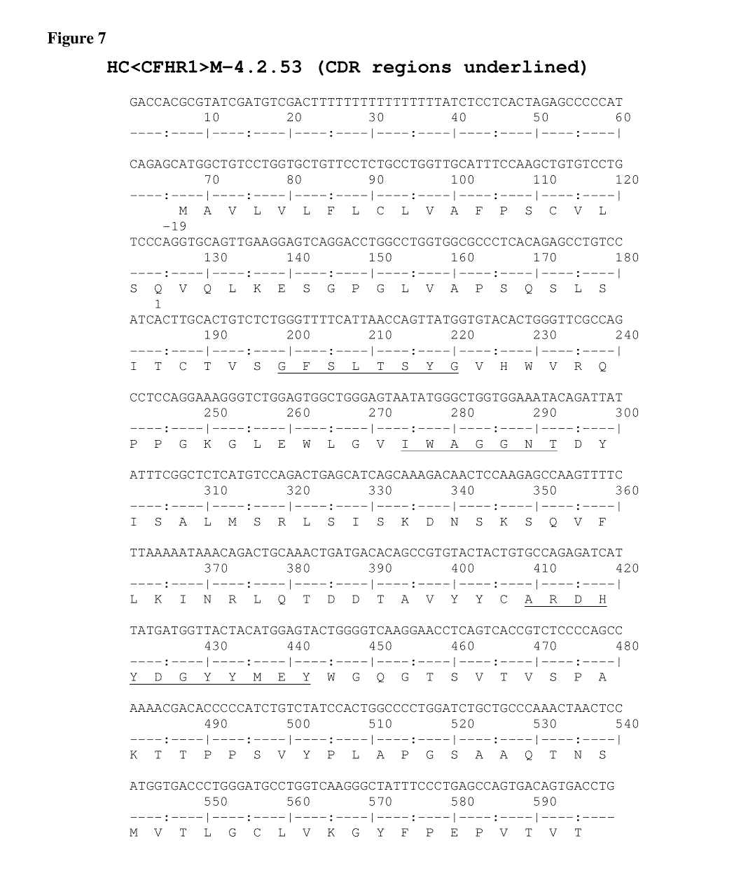

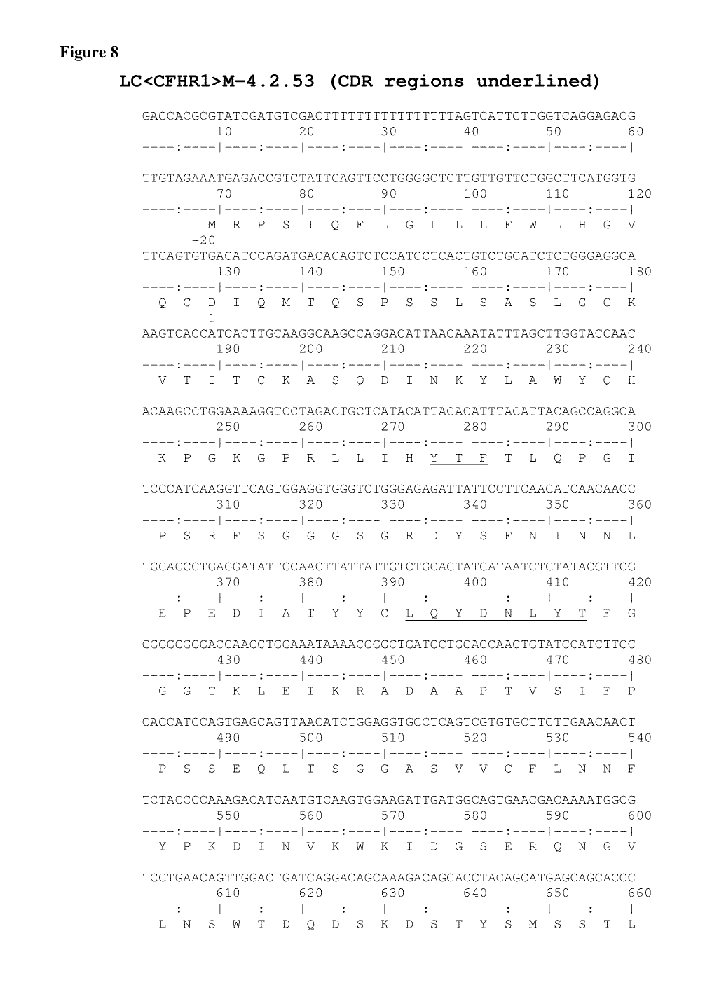

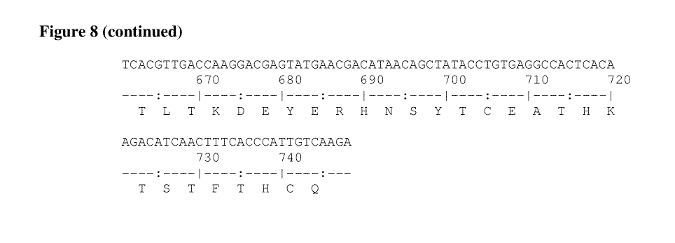

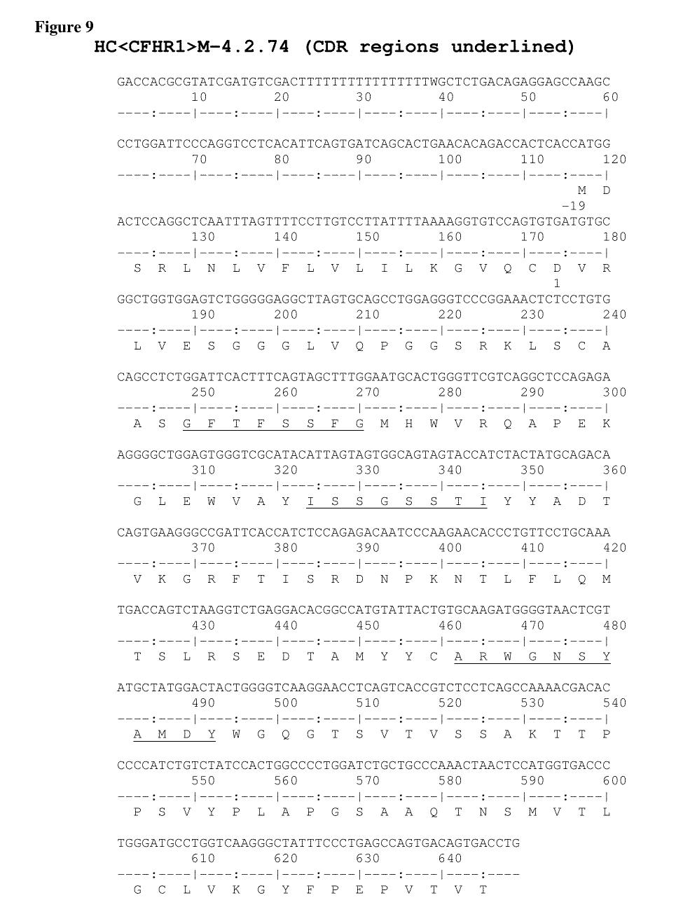

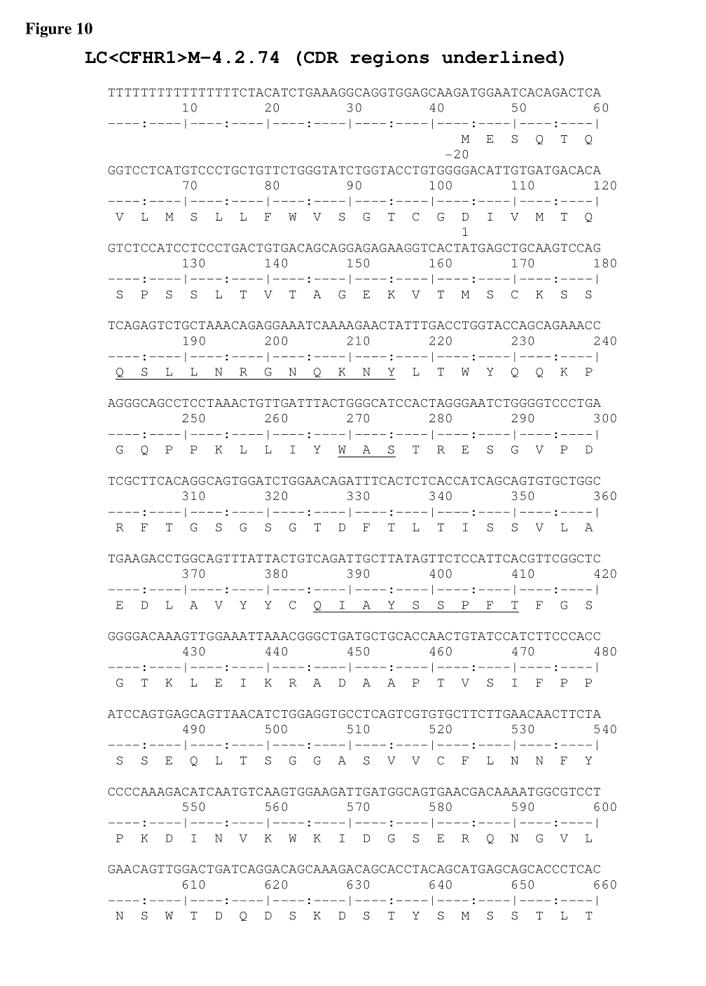

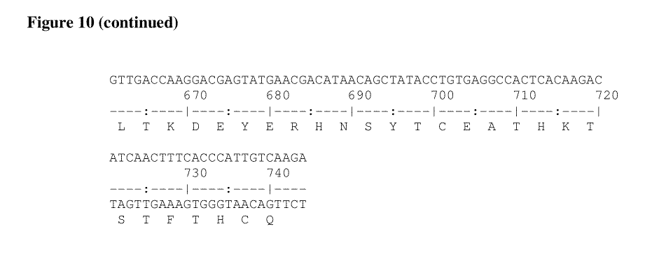

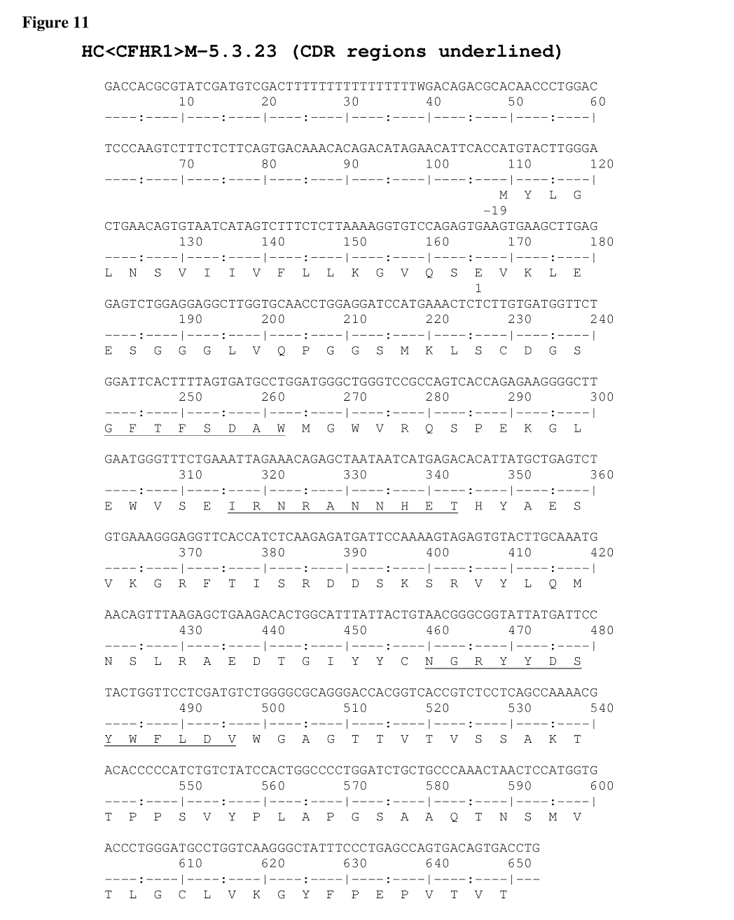

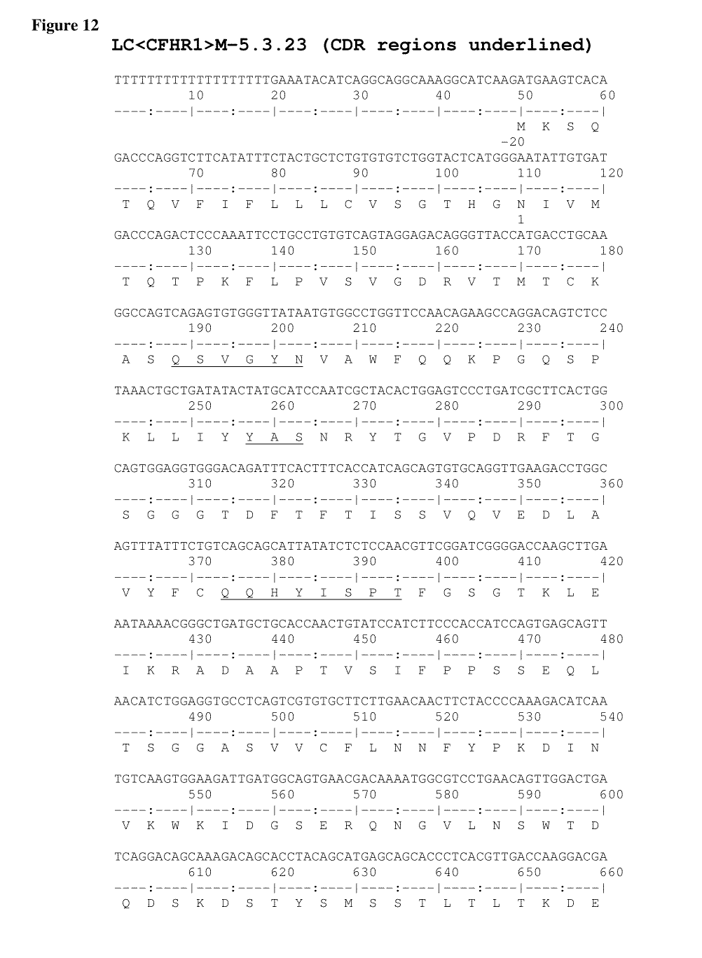

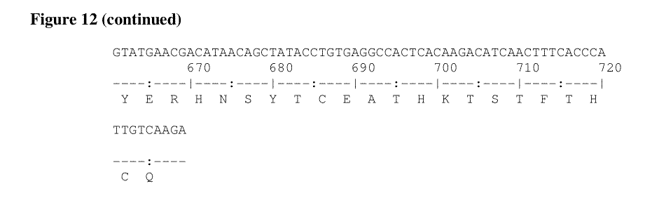

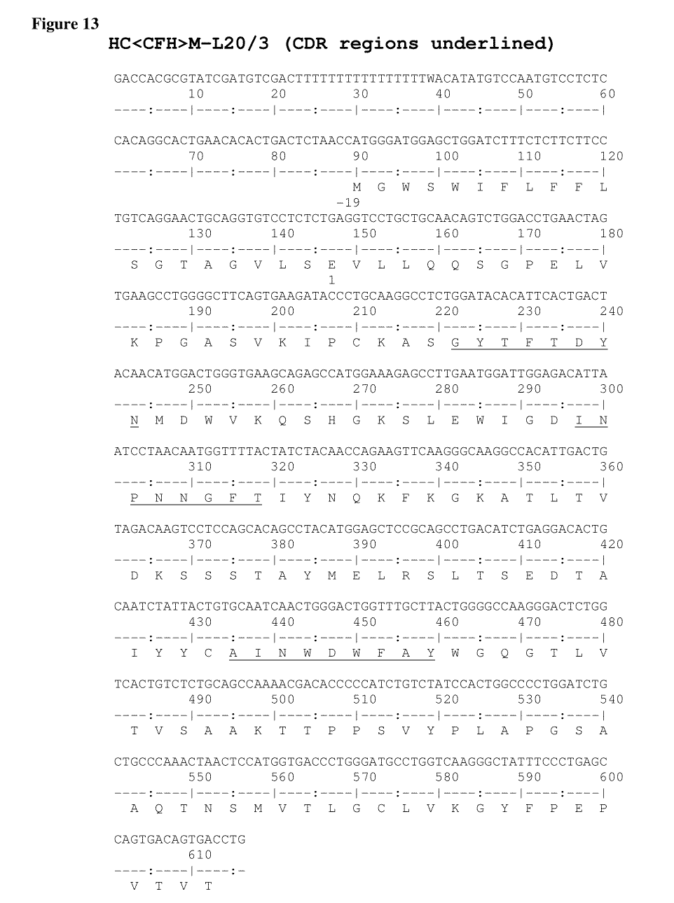

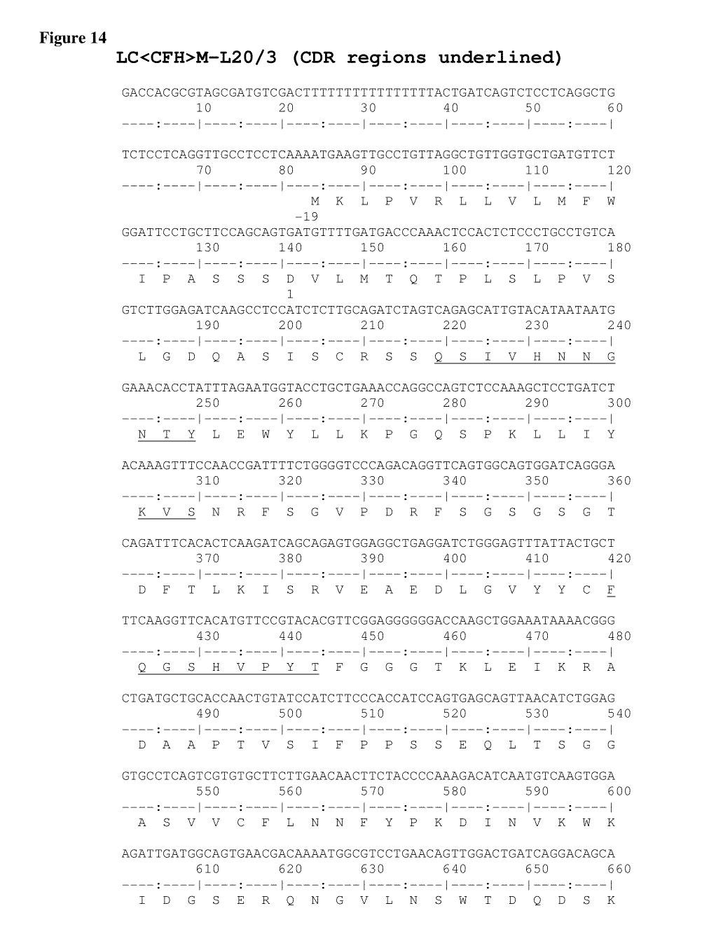

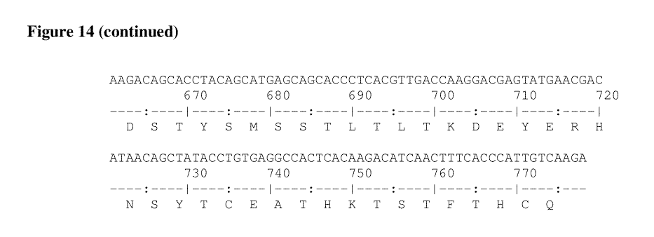

The CDR sequences of these antibodies, including the CDR3 sequences are disclosed in FIGS. 5 to 12 of the present application.

In a further embodiment, the present invention relates a functionally active variant of an agent, in particular antibody, capable of binding of CFHR1 according to the present invention.

As known to the skilled person, binding characteristics of antibodies are mediated by the variable domains. For binding to an antigen, it is essential that a suitable variable domain from the heavy chain and a co-acting variable domain from the light chain are present and arranged in order to allow for the co-acting. The variable domain is also referred to as the Fᵥ region and is the most important region for binding to antigens. More specifically variable loops, three each on the light (V_{L}) and heavy (V_{H}) chains are responsible for binding to the antigen. These loops are referred to as the Complementarity Determining Regions (CDRs). The three loops are referred to as L1, L2 and L3 for V_{L} and H1, H2 and H3 for V_{H}. However, a variety of different arrangements of variable domain from the heavy chain and a co-acting variable domain from the light chain, and CDRs of the heavy chain and CDRs of the light chain are known in the art.

A variety of different antibody formats have been developed or identified so far. Any of these or any other suitable arrangement may be used for the agent of the present invention, as long as the format or arrangement allows for binding to CFHR1.

The CDR sequences may be arranged in one polypeptide or in a peptide complex in the agents of the invention and agents for the kits of the invention. If they are arranged in one polypeptide the two sequences may be connected by a linker sequence, preferably a peptide linker, e.g. as a fusion protein. If they are arranged in a polypeptide complex, two or more polypeptides are bound to each other by non-covalent bonding including hydrogen bonds, ionic bonds, Van der Waals forces, and hydrophobic interactions. The above sequences or functionally active variants thereof may constitute the agent or may be part thereof.

A polypeptide (also known as proteins) is an organic compound made of α-amino acids arranged in a linear chain. The amino acids in a polymer chain are joined together by the peptide bonds between the carboxyl and amino groups of adjacent amino acid residues. In general, the genetic code specifies 20 standard amino acids. After or even during synthesis, the residues in a protein may be chemically modified by post-translational modification, which alter the physical and chemical properties, folding, stability, activity, and ultimately, the function of the proteins.

Agents as defined herein selectively recognize and bind to CFHR1 and are thus CFHR1-binding agents.

In specific embodiments, CFHR1-binding agents, or antibodies bind to human CFHR1 with a K_{D} of 1×10⁻⁶ or less. In specific embodiments, CFHR1-binding agents, or antibodies bind to human CFHR1 with a K_{D} of 5×10⁻⁷ or less, of 2×10⁻⁷ or less, or of 1×10⁻⁷ or less. In additional embodiments, CFHR1-binding agents bind to human CFHR1 with a K_{D} of 1×10⁻⁸ or less. In other embodiments, CFHR1-binding agents bind to human CFHR1 with a K_{D} of 5×10⁻⁹ or less, or of 1×10⁻⁹ or less. In further embodiments, CFHR1-binding agents bind to human CFHR1 with a K_{D} of 1×10⁻¹⁰ or less, a K_{D} of 1×10⁻¹¹ or less, or a K_{D} of 1×10⁻¹² or less. In specific embodiments, CFHR1-binding agents, or CFHR1-binding antibodies may bind to other proteins of the CFH protein family as described above.

The assays of the invention are specific for CFHR1. Use of the terms “selective” or “specific” herein refers to the fact that the assays of the invention do not detect other proteins of the CFH protein family, or detect other proteins of the CFH protein family with a crossreactivity of up to 0.2%, preferably up to 0.1%.

K_{D} refers to the dissociation constant obtained from the ratio of k_{d} (the dissociation rate of a particular binding molecule-target protein interaction; also referred to as k_{off}) to kₐ (the association rate of the particular binding molecule-target protein interaction; also referred to as kₒₙ), or k_{d}/kₐ which is expressed as a molar concentration (M). K_{D} values can be determined using methods well established in the art. A preferred method for determining the K_{D} of a binding molecule is by using surface plasmon resonance, for example a biosensor system such as a Biacore™ (GE Healthcare Life Sciences) system (see Example 4).

The agent may comprise also a functionally active variant of the above sequences of the antibodies of the invention. A functionally active variant of the invention is characterized by binding to CFHR1, preferably by strong binding to CFHR1.

The variant is functionally active in the context of the present invention, if the binding activity to CFHR1, optionally expressed as K_{D}, of the variant amounts to at least 10%, preferably at least 25%, more preferably at least 50%, even more preferably at least 70%, still more preferably at least 80%, especially at least 90%, particularly at least 95%, most preferably at least 99% of the activity of the agent, or antibody, without sequence alteration. Suitable methods for determining binding activity to CFHR1 are given in the Examples, as described above. A functionally active variant may be obtained by a limited number of amino acid substitutions, deletions and/or insertions.

In a preferred embodiment of the present invention the functionally active variant of SEQ ID NO: 18 comprises the complementarity determining region L3 (CDR L3), preferably CDR L1, CDR L2 and CDR L3, of the respective sequence of SEQ ID NO: 18; and/or the functionally active variant of any of the sequences SEQ ID NO: 16 comprises the complementarity determining region H3 (CDR H3), preferably CDR H1, CDR H2 and CDR H3, of the respective sequence of SEQ ID NO: 18. In a most preferred embodiment the functionally active variant of SEQ ID NO: 16 comprises CDR L1, CDR L2 and CDR L3 of the respective sequence of SEQ ID NO: 16; and the functionally active variant of the sequence SEQ ID NO: 18 comprises CDR H1, CDR H2 and CDR H3 of the respective sequence of SEQ ID NO: 18. Alternatively, one of the sequences may be SEQ ID NO: 16 or 18 without any sequence alterations and the other may be a variant as defined herein.

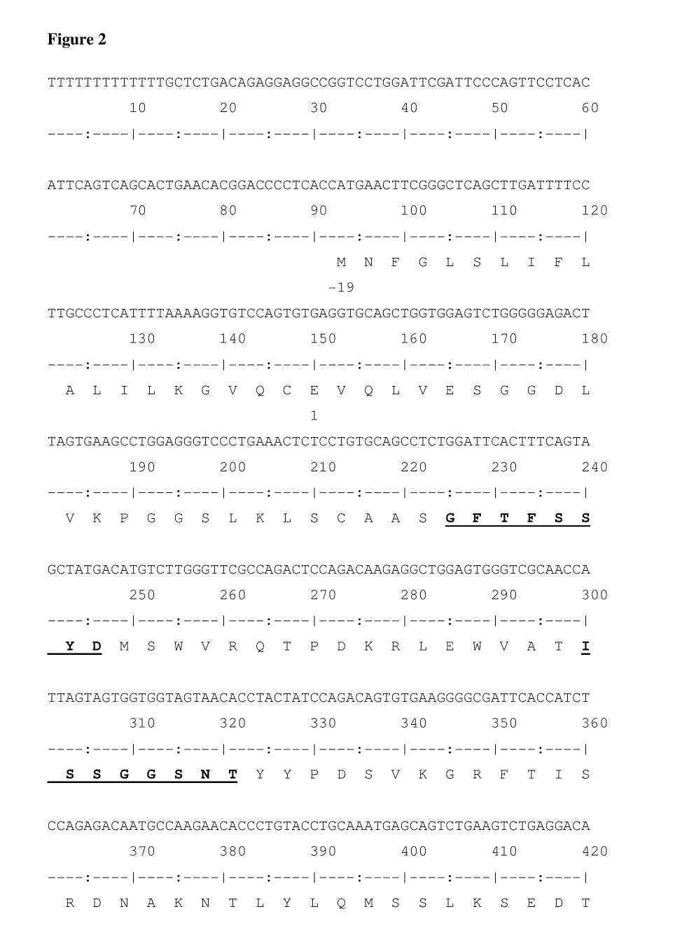

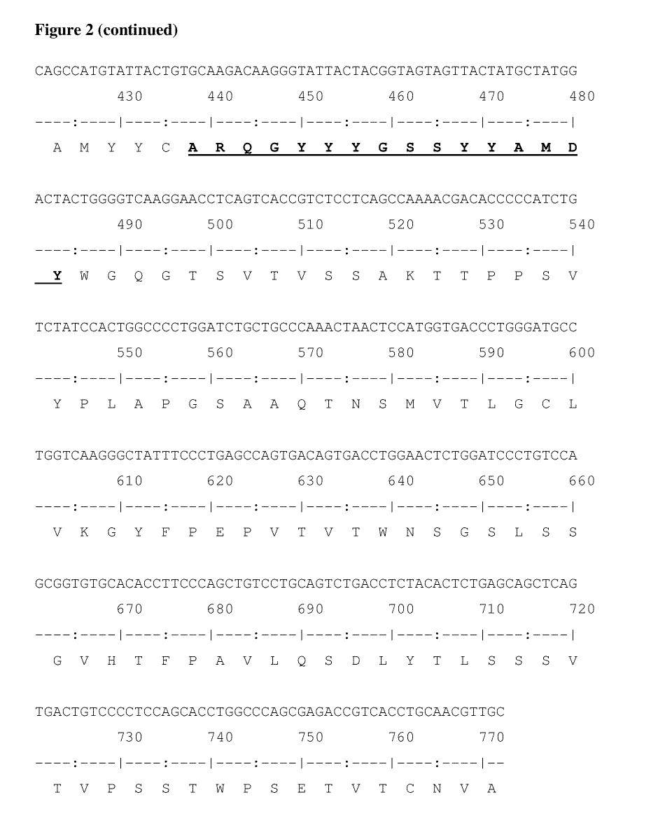

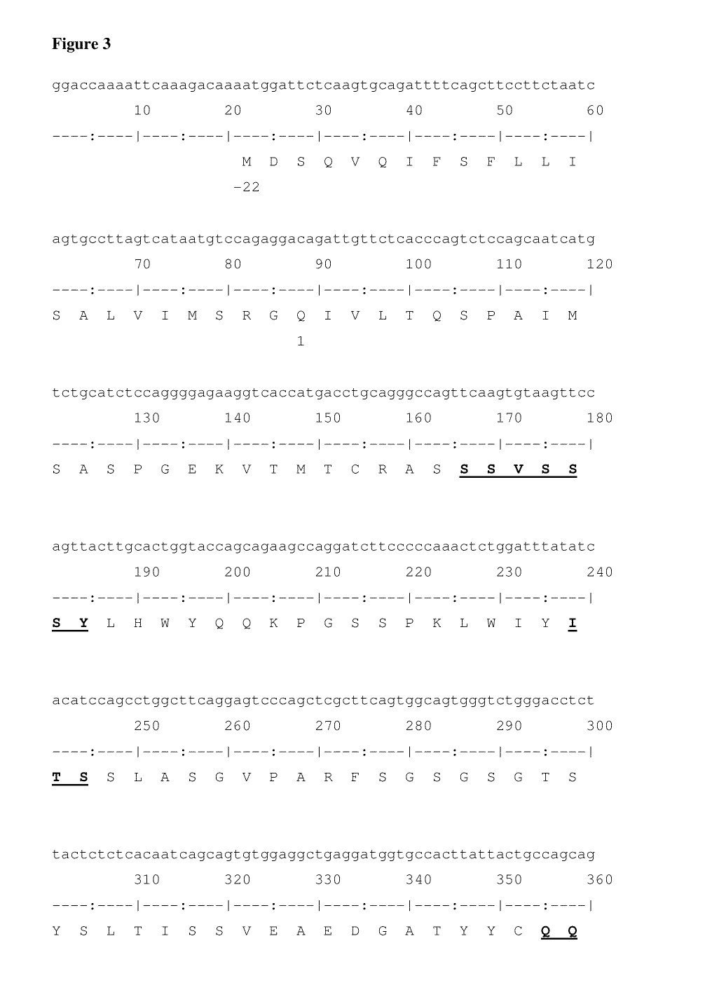

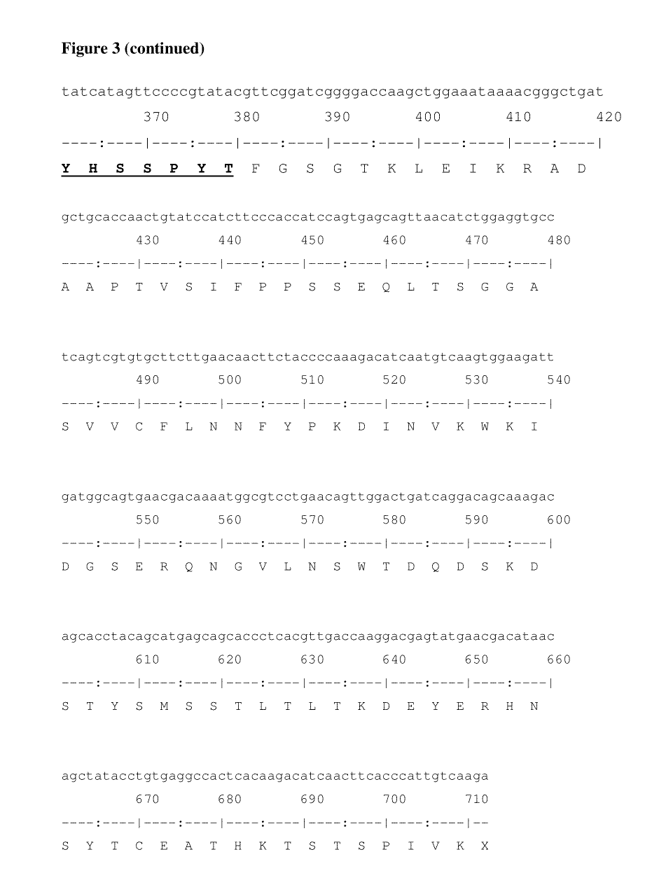

Different methods of identifying CDRs in a sequence of a variable region have been described. Additionally, a series of software programs are known, which may be used for this purpose. The set of rules which have been applied to the sequences of SEQ ID NO: 16 and 18 to identify the CDRs in these sequences are known in the art and are for example described in www.bioinf.org.uk; MacCallum et al., 1996, J. Mol. Biol. 262 (5): 732-745; Antibody Engineering Lab Manual, Chapter “Protein Sequence and Structure Analysis of Antibody Variable Domains”, Ed.: Duebel, S. and Kontermann, R., Springer-Verlag, Heidelberg. The sequences with CDRs indicated are shown in FIGS. 2 and 3.

The same applies for the functionally active variants of SEQ ID NO: 25, 27, 29, 31, 33, 35, 37 and 39.

As detailed above, within V_{H} and V_{L} there are hypervariable regions which show the most sequence variability from one antibody to another and framework regions which are less variable. Folding brings the hypervariable regions together to form the antigen-binding pockets. These sites of closest contact between antibody and antigen are the CDR of the antibody which mediates the specificity of the antibody. Accordingly, they are of particular importance for antigen binding. Though it is preferred that the functionally active variant comprises all three CDR, it has been found that for some antibodies CDR-L3 and CDR-H3 are sufficient to confer specificity. Accordingly, in one embodiment only the presence of CDR-L3 and CDR-H3 is mandatory. In any case, the CDRs have to be arranged to allow for binding to the antigen, here CFHR1.

In a preferred embodiment of the present invention the CDRs (CDR-L3 and -H3; or CDR-L1, -L2, -L3, -H1, -H2 and -H3) are arranged in the framework of the prevailing variable domain, i.e. L1, L2 and L3 in the framework of V_{L} and H1, H2 and H3 in the framework of V_{H}. This means that the CDRs as identified by any suitable method or as shown in FIGS. 2 and 3 may be removed from the shown neighborhood and transferred into another (second) variable domain, thereby substituting the CDRs of the second variable domain. Additionally, the framework of a variable domain which is not shown in FIGS. 2 and 3 may be used. A variety of variable domains or antibody sequences are known in the art and may be used for this purpose. For example, variable domains, into which CDRs of interest are inserted, may be obtained from any germ-line or rearranged human variable domain. Variable domains may also be synthetically produced. The CDR regions can be introduced into the respective variable domains using recombinant DNA technology. One means by which this can be achieved is described in Marks et al., 1992, Bio/Technology 10:779-783. A variable heavy domain may be paired with a variable light domain to provide an antigen binding site. In addition, independent regions (e.g., a variable heavy domain alone) may be used to bind antigen.

Finally, in another embodiment, the CDRs may be transferred to a non variable domain neighborhood as long as the neighborhood arranges the CDRs to allow for binding to CFHR1.

In a preferred embodiment of the present invention, the agent is an antibody.

Naturally occurring antibodies are globular plasma proteins (˜150 kDa (http://en.wikipedia.org/wiki/Dalton_unit)) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM. In the present invention, examples of suitable formats include the format of naturally occurring antibodies including antibody isotypes known as IgA, IgD, IgE, IgG and IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two beta sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids.

Each heavy chain has two regions, the constant region (C_{H}) and the variable region (V_{H}). In one species, the constant region is identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals there are two types of immunoglobulin light chain denoted by λ and κ. A light chain has two successive domains: one constant domain (C_{L}) and one variable domain (V_{L}). The approximate length of a light chain is 211 to 217 amino acids. Each antibody contains two light chains that are always identical; only one type of light chain, κ or λ, is present per antibody in mammals. Other types of light chains, such as the ι chain, are found in lower vertebrates like Chondrichthyes and Teleostei.

In addition to naturally occurring antibodies, artificial antibody formats including antibody fragments have been developed. Some of them are described in the following. However, any other antibody format comprising or consisting of the above polypeptide(s) and allowing for binding to CFHR1 are encompassed by the present invention as well.

Although the general structure of all antibodies is very similar, the unique property of a given antibody is determined by the variable (V) regions, as detailed above. More specifically, variable loops, three each the light (V_{L}) and three on the heavy (V_{H}) chain, are responsible for binding to the antigen, i.e. for its antigen specificity. These loops are referred to as the Complementarity Determining Regions (CDRs). Because CDRs from both V_{H} and V_{L} domains contribute to the antigen-binding site, it is the combination of the heavy and the light chains, and not either alone, that determines the final antigen specificity.

Accordingly, the term “antibody”, as used herein, means any polypeptide which has structural similarity to a naturally occurring antibody and is capable of binding to CFHR1, wherein the binding specificity is determined by the CDRs of the polypeptides, e.g. as shown in FIGS. 2 and 3. Hence, “antibody” is intended to relate to an immunoglobulin-derived structure with binding to CFHR1 including, but not limited to, a full length or whole antibody, an antigen binding fragment (a fragment derived, physically or conceptually, from an antibody structure), a derivative of any of the foregoing, a chimeric molecule, a fusion of any of the foregoing with another polypeptide, or any alternative structure/composition which selectively binds to CFHR1. The antibody may be any polypeptide which comprises at least one antigen binding fragment. Antigen binding fragments consist of at least the variable domain of the heavy chain and the variable domain of the light chain, arranged in a manner that both domains together are able to bind to the specific antigen.

“Full length” or “complete” antibodies refer to proteins that comprise two heavy (H) and two light (L) chains inter-connected by disulfide bonds which comprise: (1) in terms of the heavy chains, a variable region and a heavy chain constant region which comprises three domains, C_{H}1, C_{H}2 and C_{H}3; and (2) in terms of the light chains, a light chain variable region and a light chain constant region which comprises one domain, C_{L}. With regard to the term “complete antibody”, any antibody is meant that has a typical overall domain structure of a naturally occurring antibody (i.e. comprising a heavy chain of three or four constant domains and a light chain of one constant domain as well as the respective variable domains), even though each domain may comprise further modifications, such as mutations, deletions, or insertions, which do not change the overall domain structure. For instance, MAB<CFHR1>M-5.1.5, is a full length antibody.

An “antibody fragment” also contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)₂ fragment containing both Fab pieces and the hinge region, including the H-H interchain disulfide bond. F(ab′)₂ is divalent for antigen binding. The disulfide bond of F(ab′)₂ may be cleaved in order to obtain Fab′. Moreover, the variable regions of the heavy and light chains can be fused together to form a single chain variable fragment (scFv).

As the first generation of full sized antibodies presented some problems, many of the second generation antibodies have comprised only fragments of the antibody. Variable domains (Fvs) are the smallest fragments with an intact antigen-binding domain consisting of one V_{L} and one V_{H}. Such fragments, with only the binding domains, can be generated by enzymatic approaches or expression of the relevant gene fragments, e.g. in bacterial and eukaryotic cells. Different approaches can be used, e.g. either the Fv fragment alone or ‘Fab’-fragments comprising one of the upper arms of the “Y” that includes the Fv plus the first constant domains. These fragments are usually stabilized by introducing a polypeptide link between the two chains which results in the production of a single chain Fv (scFv). Alternatively, disulfide-linked Fv (dsFv) fragments may be used. The binding domains of fragments can be combined with any constant domain in order to produce full length antibodies or can be fused with other proteins and polypeptides.

A recombinant antibody fragment is the single-chain Fv (scFv) fragment. In general, it has a high affinity for its antigen and can be expressed in a variety of hosts. These and other properties make scFv fragments not only applicable in medicine, but also of potential for biotechnological applications. As detailed above, in the scFv fragment the V_{H} and V_{L} domains are joined with a hydrophilic and flexible peptide linker, which improves expression and folding efficiency. Usually linkers of about 15 amino acids are used, of which the (Gly₄Ser)₃ linker has been used most frequently. scFv molecules might be easily proteolytically degraded, depending on the linker used. With the development of genetic engineering techniques these limitations could be practically overcome by research focussed on improvement of function and stability. An example is the generation of disulfide-stabilized (or disulfide-linked) Fv fragments where the V_{H}-V_{L} dimer is stabilized by an interchain disulfide bond. Cysteines are introduced at the interface between the V_{L} and V_{H} domains, forming a disulfide bridge, which holds the two domains together.

Dissociation of scFvs results in monomeric scFvs, which can be complexed into dimers (diabodies), trimers (triabodies) or larger aggregates such as TandAbs and Flexibodies.

Antibodies with two binding domains can be created either through the binding of two scFv with a simple polypeptide link (scFv)2 or through the dimerization of two monomers (diabodies). The simplest designs are diabodies that have two functional antigen-binding domains that can be either the same, similar (bivalent diabodies) or have specificity for distinct antigens (bispecific diabodies). These bispecific antibodies allow for example the recruitment of novel effector functions (such as cytotoxic T cells) to the target cells, which make them very useful for applications in medicine.

Recently, antibody formats comprising four variable domains of heavy chains and four variable domains of light chains have been developed. Examples of these include tetravalent bispecific antibodies (TandAbs and Flexibodies, Affimed Therapeutics AG, Heidelberg. Germany). In contrast to a bispecific diabody, a bispecific TandAb is a homodimer consisting of only one polypeptide. Because the two different chains, a diabody can build three different dimers only one of which is functional. Therefore, it is simpler and cheaper to produce and purify this homogeneous product. Moreover, the TandAb usually shows better binding properties (possessing twice the number of binding sites) and increased stability in vivo. Flexibodies are a combination of scFv with a diabody multimer motif resulting in a multivalent molecule with a high degree of flexibility for joining two molecules which are quite distant from each other on the cell surface. If more than two functional antigen-binding domains are present and if they have specificity for distinct antigens, the antibody is multispecific.

In summary, specific immunoglobulins, into which particular disclosed sequences may be inserted or, in the alternative, form the essential part of, include but are not limited to the following antibody molecules which form particular embodiments of the present invention: a Fab (monovalent fragment with variable light (V_{L}), variable heavy (V_{H}), constant light (C_{L}) and constant heavy 1 (C_{H}1) domains), a F(ab′)2 (bivalent fragment comprising two Fab fragments linked by a disulfide bridge or alternative at the hinge region), a Fv (V_{L} and V_{H} domains), a scFv (a single chain Fv where V_{L} and V_{H} are joined by a linker, e.g., a peptide linker), a bispecific antibody molecule (an antibody molecule comprising a polypeptide as disclosed herein linked to a second functional moiety having a different binding specificity than the antibody, including, without limitation, another peptide or protein such as an antibody, or receptor ligand), a bispecific single chain Fv dimer, a diabody, a triabody, a tetrabody, a minibody (a scFv joined to a C_{H}3).

Certain antibody molecules including, but not limited to, Fv, scFv, diabody molecules or domain antibodies (Domantis) may be stabilized by incorporating disulfide bridges to line the VH and VL domains. Bispecific antibodies may be produced using conventional technologies, specific methods of which include production chemically, or from hybrid hybridomas) and other technologies including, but not limited to, the BiTE™ technology (molecules possessing antigen binding regions of different specificity with a peptide linker) and knobs-into-holes engineering.

Accordingly, the antibody may be a Fab, a Fab′, a F(ab′)2, a Fv, a disulfide-linked Fv, a scFv, a (scFv)₂, a bivalent antibody, a bispecific antibody, a multispecific antibody, a diabody, a triabody, a tetrabody or a minibody.

In another preferred embodiment, the antibody is a monoclonal antibody, a chimeric antibody or a humanised antibody. Monoclonal antibodies are monospecific antibodies that are identical because they are produced by one type of immune cell that are all clones of a single parent cell. A chimeric antibody is an antibody in which at least one region of an immunoglobulin of one species is fused to another region of an immunoglobulin of another species by genetic engineering in order to reduce its immunogenecity. For example murine V_{L} and V_{H} regions may be fused to the remaining part of a human immunoglobulin. A particular type of chimeric antibodies are humanised antibodies. Humanised antibodies are produced by merging the DNA that encodes the CDRs of a non-human antibody with human antibody-producing DNA. The resulting DNA construct can then be used to express and produce antibodies that are usually not as immunogenic as the non-human parenteral antibody or as a chimeric antibody, since merely the CDRs are non-human.

In a preferred embodiment of the present invention, the agent comprises a heavy chain immunoglobulin constant domain selected from the group consisting of: a human IgM constant domain, a human IgG1 constant domain, a human IgG2 constant domain, a human IgG3 constant domain, a human IgG4 constant domain, a human IgE constant domain, and a human IgA constant domain.

As detailed above in the context with the antibody of the present invention, each heavy chain of a naturally occurring antibody has two regions, the constant region and the variable region. There are five types of mammalian immunoglobulin heavy chain: γ, δ, α, μ and ε, which define classes of immunoglobulins IgM, IgD, IgG, IgA and IgE, respectively.

There are here are four IgG subclasses (IgG1, 2, 3 and 4) in humans, named in order of their abundance in serum (IgG1 being the most abundant). Even though there is about 95% similarity between their Fc regions of the IgG subclasses, the structure of the hinge regions are relatively different. This region, between the Fab arms (Fragment antigen binding) and the two carboxy-terminal domains C_{H}2 and C_{H}3 of both heavy chains, determines the flexibility of the molecule. The upper hinge (towards the amino-terminal) segment allows variability of the angle between the Fab arms (Fab-Fab flexibility) as well as rotational flexibility of each individual Fab. The flexibility of the lower hinge region (towards the carboxy-terminal) directly determines the position of the Fab-arms relative to the Fc region (Fab-Fc flexibility). Hinge-dependent Fab-Fab and Fab-Fc flexibility may be important in triggering further effector functions such as complement activation and Fc receptor binding. Accordingly, the structure of the hinge regions gives each of the four IgG classes their unique biological profile.

The length and flexibility of the hinge region varies among the IgG subclasses. The hinge region of IgG1 encompasses amino acids 216-231 and since it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, it is relatively short and contains a rigid poly-proline double helix, stabilised by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3 the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2.

In a preferred embodiment of the present invention, the present invention relates to a functionally active variant of monoclonal antibody MAB<CFHR1>M-5.1.5, wherein the heavy chain has a sequence of SEQ ID No. 16, and wherein the light chain has a sequence of SEQ ID No. 18, or of an antibody comprising the CDR sequences of MAB<CFHR1>M-5.1.5 according to SEQ ID No. 19, SEQ ID No. 20, SEQ ID No. 21, SEQ ID No. 22, and SEQ ID No. 23 and the amino acid sequence ITS and/or comprising the CDR3 sequences of MAB<CFHR1>M-5.1.5 according to SEQ ID No. 21 and SEQ ID No. 23.

The invention further relates in another preferred embodiment to a functionally active variant of monoclonal antibody MAB<CFHR1>M-4.1.3, wherein the heavy chain has a sequence of SEQ ID No. 25, and wherein the light chain has a sequence of SEQ ID No. 27, or of monoclonal antibody MAB<CFHR1>M-4.2.53, wherein the heavy chain has a sequence of SEQ ID No. 29, and wherein the light chain has a sequence of SEQ ID No. 31, or of monoclonal antibody MAB<CFHR1>M-4.2.74, wherein the heavy chain has a sequence of SEQ ID No. 33, and wherein the light chain has a sequence of SEQ ID No. 35, or of monoclonal antibody MAB<CFHR1>M-5.3.23, wherein the heavy chain has a sequence of SEQ ID No. 37, and wherein the light chain has a sequence of SEQ ID No. 39, or of an antibody comprising the CDR sequences of MAB<CFHR1>M-4.1.3, MAB<CFHR1>M-4.2.53, MAB<CFHR1>M-4.2.74, or MAB<CFHR1>M-5.3.23 and/or comprising the CDR3 sequences of MAB<CFHR1>M-4.1.3, MAB<CFHR1>M-4.2.53, MAB<CFHR1>M-4.2.74, or MAB<CFHR1>M-5.3.23.

For example, the variant may be defined in that the variant
a) is a functionally active fragment consisting of at least 60%, preferably at least 70%, more preferably at least 80%, still more preferably at least 90%, even more preferably at least 95%, most preferably 99% of an amino acid sequence of any of the SEQ ID NOS: 16, 18, 25, 27, 29, 31, 33, 35, 37 and/or 39;
b) is a functionally active variant having at least 60%, preferably at least 70%, more preferably at least 80%, still more preferably at least 90%, even more preferably at least 95%, most preferably 99% sequence identity to an amino acid sequence of any of the SEQ ID NOS: 16, 18, 25, 27, 29, 31, 33, 35, 37 and/or 39; or
c) consists of an amino acid sequence of any of the SEQ ID NOS: 16, 18, 25, 27, 29, 31, 33, 35, 37 and/or 39 and 1 to 50 additional amino acid residue(s), preferably 1 to 40, more preferably 1 to 30, even more preferably at most 1 to 25, still more preferably at most 1 to 10, most preferably 1, 2, 3, 4 or 5 additional amino acids residue(s).

The fragment as defined in a) is characterized by being derived from any of the sequences of SEQ ID NO: 16, 18, 25, 27, 29, 31, 33, 35, 37 and 39 by one or more deletions. The deletion(s) may be C-terminally, N-terminally and/or internally. Preferably the fragment is obtained by 1, 2, 3, 4, 5, 6, 7, 8, 9 or 10, more preferably 1, 2, 3, 4 or 5, even more preferably 1, 2 or 3, still more preferably 1 or 2, most preferably 1 deletion(s). The functionally active fragment of the invention is characterized by the ability to bind to CFHR1. The fragment of an antigen is functionally active in the context of the present invention, if the binding of the fragment amounts to at least 10%, preferably at least 25%, more preferably at least 50%, even more preferably at least 70%, still more preferably at least 80%, especially at least 90%, particularly at least 95%, most preferably at least 99% of the activity of the antigen without sequence alteration.

The variant as defined in b) is characterized by being derived from any of the sequences of SEQ ID NO: 16, 18, 25, 27, 29, 31, 33, 35, 37 and 39 by one or more amino acid modifications including deletions, additions and/or substitutions. The modification(s) may be C-terminally, N-terminally and/or internally. Preferably the fragment is obtained by 1, 2, 3, 4, 5, 6, 7, 8, 9 or 10, more preferably 1, 2, 3, 4 or 5, even more preferably 1, 2 or 3, still more preferably 1 or 2, most preferably 1 modification(s). The functionally active variant of the invention is characterized by the ability to bind to CFHR1. The fragment of an antigen is functionally active in the context of the present invention, if the binding of the fragment amounts to at least 10%, preferably at least 25%, more preferably at least 50%, even more preferably at least 70%, still more preferably at least 80%, especially at least 90%, particularly at least 95%, most preferably at least 99% of the activity of the antigen without sequence alteration.

The variant as defined in c) is characterized in that it consists of an amino acid sequence of any of the SEQ ID NOS: 16, 18, 25, 27, 29, 31, 33, 35, 37 or 39 and 1 to 50 additional amino acid residue(s). The addition(s) may be C-terminally, N-terminally and/or internally. Preferably the variant is obtained by 1, 2, 3, 4, 5, 6, 7, 8, 9 or 10, more preferably 1, 2, 3, 4 or 5, even more preferably 1, 2 or 3, still more preferably 1 or 2, most preferably 1 addition(s). The functionally active variant is further defined as above (see variant of b)).

The additional amino acid residue(s) of (b) and/or (c) may be any amino acid, which may be either an L- and/or a D-amino acid, naturally occurring and other. Preferably, the amino acid is any naturally occurring amino acid such as alanine, cysteine, aspartic acid, glutamic acid, phenylalanine, glycine, histidine, isoleucine, lysine, leucine, methionine, asparagine, proline, glutamine, arginine, serine, threonine, valine, tryptophan or tyrosine.

However, the amino acid may also be a modified or unusual amino acid. Examples of those are 2-aminoadipic acid, 3-aminoadipic acid, beta-alanine, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminobutyric acid, desmosine, 2,2′-diaminopimelic acid, 2,3-diaminopropionic acid, N-ethylglycinem N-ethylasparagine, hydroxylysine, allo-hydroxylysine, 3-hydroxyproloine, 4-hydroxyproloine, isodesmosine, allo-isoleucine, N-methylglycine, N-methylisoleucine, 6-N-Methyllysine, N-methylvaline, norvaline, norleucine or ornithine.

Additionally, the amino acid may be subject to modifications such as posttranslational modifications. Examples of modifications include acetylation, amidation, blocking, formylation, γ-carboxyglutamic acid hydroxylation, glycosilation, methylation, phosphorylation and sulfatation. If more than one additional or heterologous amino acid residue is present in the peptide, the amino acid residues may be the same or different from one another.

The percentage of sequence identity can be determined e.g. by sequence alignment. Methods of alignment of sequences for comparison are well known in the art. Various programs and alignment algorithms have been described e.g. in Smith and Waterman, Adv. Appl. Math. 2: 482, 1981 or Pearson and Lipman, Proc. Natl. Acad. Sci. US.A. 85: 2444, 1988.

The NCBI Basic Local Alignment Search Tool (BLAST) (Altschul et al., J. Mol. Biol. 215: 403-410, 1990) is available from several sources, including the National Center for Biotechnology Information (NCBI, Bethesda, Md.) and on the Internet, for use in connection with the sequence analysis programs blastp, blastn, blastx, tblastn and tblastx. Variants of any of the sequences of SEQ ID NOS: 16 and 18 are typically characterized using the NCBI Blast 2.0, gapped blastp set to default parameters. For comparisons of amino acid sequences of at least 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment is performed using the Blast 2 sequences function, employing the PAM30 matrix set t default parameters (open gap 9, extension gap 1 penalties). Methods for determining sequence identity over such short windows such as 15 amino acids or less are described at the website that is maintained by the National Center for Biotechnology Information in Bethesda, Md.

In a more preferred embodiment the functionally active variant, as defined above, is derived from the amino acid sequence of any of the SEQ ID NOS: 16 and 18 by one or more conservative amino acid substitution.

Conservative amino acid substitutions, as one of ordinary skill in the art will appreciate, are substitutions that replace an amino acid residue with one imparting similar or better (for the intended purpose) functional and/or chemical characteristics. For example, conservative amino acid substitutions are often ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Such modifications are not designed to significantly reduce or alter the binding or functional inhibition characteristics of the agent, albeit they may improve such properties. The purpose for making a substitution is not significant and can include, but is by no means limited to, replacing a residue with one better able to maintain or enhance the structure of the molecule, the charge or hydrophobicity of the molecule, or the size of the molecule. For instance, one may desire simply to substitute a less desired residue with one of the same polarity or charge. Such modifications can be introduced by standard techniques known in the art, such as site-directed mutagenesis and PCR-mediated mutagenesis. One specific means by which those of skill in the art accomplish conservative amino acid substitutions is alanine scanning mutagenesis. The altered polypeptides are then tested for retained or better function using functional assays available in the art or described in the Examples. In a more preferred embodiment of the present invention the number of conservative substitutions in any of the sequences of SEQ ID NO: 16, 18, 25, 27, 29, 31, 33, 35, 37 or 39 is at most 20, 19, 18, 27, 26, 15, 14, 13, 12 or 11, preferably at most 10, 9, 8, 7 or 6, especially at most 5, 4, 3 particularly 2 or 1.

In a further embodiment, the present invention relates to one or more nucleic acid(s) coding for an agent, in particular antibody, of the present invention.

Nucleic acid molecules of the present invention may be in the form of RNA, such as mRNA or cRNA, or in the form of DNA, including, for instance, cDNA and genomic DNA e.g. obtained by cloning or produced by chemical synthetic techniques or by a combination thereof. The DNA may be triple-stranded, double-stranded or single-stranded. Single-stranded DNA may be the coding strand, also known as the sense strand, or it may be the non-coding strand, also referred to as the anti-sense strand. Nucleic acid molecule as used herein also refers to, among other, single- and double-stranded DNA, DNA that is a mixture of single- and double-stranded RNA, and RNA that is a mixture of single- and double-stranded regions, hybrid molecules comprising DNA and RNA that may be single-stranded or, more typically, double-stranded, or triple-stranded, or a mixture of single- and double-stranded regions. In addition, nucleic acid molecule as used herein refers to triple-stranded regions comprising RNA or DNA or both RNA and DNA.

The nucleic acid also includes sequences that are a result of the degeneration of the genetic code. There are 20 natural amino acids, most of which are specified by more than one codon. Therefore, all nucleotide sequences are included in the invention which result in the peptide(s) as defined above.

Additionally, the nucleic acid may contain one or more modified bases. Such nucleic acids may also contain modifications e.g. in the ribose-phosphate backbone to increase stability and half life of such molecules in physiological environments. Thus, DNAs or RNAs with backbones modified for stability or for other reasons are “nucleic acid molecule” as that feature is intended herein. Moreover, DNAs or RNAs comprising unusual bases, such as inosine, or modified bases, such as tritylated bases, to name just two examples, are nucleic acid molecule within the context of the present invention. It will be appreciated that a great variety of modifications have been made to DNA and RNA that serve many useful purposes known to those of skill in the art. The term nucleic acid molecule as it is employed herein embraces such chemically, enzymatically or metabolically modified forms of nucleic acid molecule, as well as the chemical forms of DNA and RNA characteristic of viruses and cells, including simple and complex cells, inter alia. For example, nucleotide substitutions can be made which do not affect the polypeptide encoded by the nucleic acid, and thus any nucleic acid molecule which encodes an antigen or fragment or functional active variant thereof as defined above is encompassed by the present invention.

Furthermore, any of the nucleic acid molecules encoding one or more agents of the invention including fragments or functionally active variants thereof can be functionally linked, using standard techniques such as standard cloning techniques, to any desired regulatory sequence, leader sequence, heterologous marker sequence or a heterologous coding sequence to create a fusion protein.

The nucleic acid of the invention may be originally formed in vitro or in a cell in culture, in general, by the manipulation of nucleic acids by endonucleases and/or exonucleases and/or polymerases and/or ligases and/or recombinases or other methods known to the skilled practitioner to produce the nucleic acids.

In a preferred embodiment, the nucleic acid(s) is/are located in a vector. A vector may additionally include nucleic acid sequences that permit it to replicate in the host cell, such as an origin of replication, one or more therapeutic genes and/or selectable marker genes and other genetic elements known in the art such as regulatory elements directing transcription, translation and/or secretion of the encoded protein. The vector may be used to transduce, transform or infect a cell, thereby causing the cell to express nucleic acids and/or proteins other than those native to the cell. The vector optionally includes materials to aid in achieving entry of the nucleic acid into the cell, such as a viral particle, liposome, protein coating or the like. Numerous types of appropriate expression vectors are known in the art for protein expression, by standard molecular biology techniques. Such vectors are selected from among conventional vector types including insects, e.g., baculovirus expression, or yeast, fungal, bacterial or viral expression systems. Other appropriate expression vectors, of which numerous types are known in the art, can also be used for this purpose. Methods for obtaining such expression vectors are well-known (see, e.g. Sambrook et al, Molecular Cloning. A Laboratory Manual, 2d edition, Cold Spring Harbor Laboratory, New York (1989)). In one embodiment, the vector is a viral vector. Viral vectors include, but are not limited to, retroviral and adenoviral vectors.

Suitable host cells or cell lines for transfection by this method include bacterial cells. For example, the various strains of E. coli are well-known as host cells in the field of biotechnology. Various strains of B. subtilis, Pseudomonas, Streptomyces, and other bacilli and the like may also be employed in this method. Many strains of yeast cells known to those skilled in the art are also available as host cells for expression of the peptides of the present invention. Other fungal cells or insect cells such as Spodoptera frugipedera (Sf9) cells may also be employed as expression systems. Alternatively, mammalian cells, such as human 293 cells, Chinese hamster ovary cells (CHO), the monkey COS-1 cell line or murine 3T3 cells derived from Swiss, BALB/c or NIH mice may be used. Still other suitable host cells, as well as methods for transfection, culture, amplification, screening, production, and purification are known in the art.

The cDNA sequences encoding the heavy and light chains of MAB<CFHR1>M-5.1.5 are SEQ ID No. 15 and 17, respectively. Thus, in a preferred embodiment, the present invention relates to a nucleic acid according to SEQ ID No. 15. In a further preferred embodiment, the present invention relates to a nucleic acid according to SEQ ID No. 17. In a preferred embodiment, the present invention relates to a nucleic acid according to SEQ ID No. 17 or SEQ ID No. 15 wherein the nucleic acid is located in a vector. In a further preferred embodiment, the present invention relates to a nucleic acid according to SEQ ID No. 17 and SEQ ID No. 15 wherein the nucleic acids is located in a vector. The nucleic acid according to SEQ ID No. 17 and SEQ ID No. 15 may be located in the same or different vectors.

The cDNA sequences encoding the heavy and light chains of MAB<CFHR1>M-4.1.3 are SEQ ID No. 24 and 26, respectively. Thus, in a preferred embodiment, the present invention relates to a nucleic acid according to SEQ ID No. 24. In a further preferred embodiment, the present invention relates to a nucleic acid according to SEQ ID No. 26. In a preferred embodiment, the present invention relates to a nucleic acid according to SEQ ID No. 24 or SEQ ID No. 26 wherein the nucleic acid is located in a vector. In a further preferred embodiment, the present invention relates to a nucleic acid according to SEQ ID No. 24 and SEQ ID No. 26 wherein the nucleic acids is located in a vector. The nucleic acid according to SEQ ID No. 24 and SEQ ID No. 26 may be located in the same or different vectors.

The cDNA sequences encoding the heavy and light chains of MAB<CFHR1>M-4.2.53 are SEQ ID No. 28 and 30, respectively. Thus, in a preferred embodiment, the present invention relates to a nucleic acid according to SEQ ID No. 28. In a further preferred embodiment, the present invention relates to a nucleic acid according to SEQ ID No. 30. In a preferred embodiment, the present invention relates to a nucleic acid according to SEQ ID No. 28 or SEQ ID No. 30 wherein the nucleic acid is located in a vector. In a further preferred embodiment, the present invention relates to a nucleic acid according to SEQ ID No. 28 and SEQ ID No. 30 wherein the nucleic acids is located in a vector. The nucleic acid according to SEQ ID No. 28 and SEQ ID No. 30 may be located in the same or different vectors.

The cDNA sequences encoding the heavy and light chains of MAB<CFHR1>M-4.2.74 are SEQ ID No. 32 and 34, respectively. Thus, in a preferred embodiment, the present invention relates to a nucleic acid according to SEQ ID No. 32. In a further preferred embodiment, the present invention relates to a nucleic acid according to SEQ ID No. 34. In a preferred embodiment, the present invention relates to a nucleic acid according to SEQ ID No. 32 or SEQ ID No. 34 wherein the nucleic acid is located in a vector. In a further preferred embodiment, the present invention relates to a nucleic acid according to SEQ ID No. 32 and SEQ ID No. 34 wherein the nucleic acids is located in a vector. The nucleic acid according to SEQ ID No. 32 and SEQ ID No. 34 may be located in the same or different vectors.

The cDNA sequences encoding the heavy and light chains of MAB<CFHR1>M-5.3.23 are SEQ ID No. 36 and 38, respectively. Thus, in a preferred embodiment, the present invention relates to a nucleic acid according to SEQ ID No. 36. In a further preferred embodiment, the present invention relates to a nucleic acid according to SEQ ID No. 38. In a preferred embodiment, the present invention relates to a nucleic acid according to SEQ ID No. 36 or SEQ ID No. 38 wherein the nucleic acid is located in a vector. In a further preferred embodiment, the present invention relates to a nucleic acid according to SEQ ID No. 36 and SEQ ID No. 38 wherein the nucleic acids is located in a vector. The nucleic acid according to SEQ ID No. 36 and SEQ ID No. 38 may be located in the same or different vectors.

An agent, in particular antibodies of the invention, may be produced by expressing a nucleic acid of the invention in a suitable host cell. The host cells can be transfected, e.g. by conventional means such as electroporation with at least one expression vector containing a nucleic acid of the invention under the control of a transcriptional regulatory sequence. The transfected or transformed host cell is then cultured under conditions that allow expression of the protein. The expressed protein is recovered, isolated, and optionally purified from the cell (or from the culture medium, if expressed extracellularly) by appropriate means known to one of skill in the art. For example, the proteins are isolated in soluble form following cell lysis, or extracted using known techniques, e.g. in guanidine chloride. If desired, the agent(s) of the invention are produced as a fusion protein. Such fusion proteins are those described above. Alternatively, for example, it may be desirable to produce fusion proteins to enhance expression of the protein in a selected host cell or to improve purification. The molecules comprising the agents of this invention may be further purified using any of a variety of conventional methods including, but not limited to: liquid chromatography such as normal or reversed phase, using HPLC, FPLC and the like; affinity chromatography (such as with inorganic ligands or monoclonal antibodies); size exclusion chromatography; immobilized metal chelate chromatography; gel electrophoresis; and the like. One of skill in the art may select the most appropriate isolation and purification techniques without departing from the scope of this invention. Such purification provides the antigen in a form substantially free from other proteinaceous and non-proteinaceous materials of the microorganism.

The invention further relates to a cell line producing an agent, in particular antibody, capable of binding CFHR1 of the present invention. In case of a monoclonal antibody, the cell line is preferably a hybridoma cell line.

In order to employ an agent, in particular antibody, capable of binding of CFHR1 in an assay of the present invention, the antibody is either labeled with a detectable label, and/or is capable of immobilizing on a solid phase. Therefore, in a further embodiment, the present invention relates an agent, in particular antibody, capable of binding of binding CFHR1 of the present invention, which antibody is labeled with a detectable label, and/or is capable of immobilizing on a solid phase.

The agents, in particular antibodies, are useful for the diagnosis of diseases and disorders, in particular CFHR1-related diseases or disorders. Therefore, in a further embodiment, the present invention relates to an agent, in particular antibody, capable of binding CFHR1, and/or one or more nucleic acid(s) of the present invention, and/or a cell line of the present invention, for use in diagnosis of a disease or disorder, in particular a CFHR1-related disease or disorder, more preferably schizophrenia.

In a further embodiment, the present invention relates to an agent, in particular antibody, capable of binding CFHR1, and/or one or more nucleic acid(s) of the present invention, and/or a cell line of the present invention, for use in treatment and/or prevention of a disease or disorder, in particular a CFHR1-related disease or disorder, more preferably schizophrenia.

In a yet further embodiment, the in vitro use of an agent, in particular of an antibody, capable of binding of CFHR1, and/or one or more nucleic acid(s) of the present invention, and/or a cell line of the present invention,
a) for determining the amount and/or concentration of CFHR1 in a sample obtained from a subject, and/or
b) for the prediction of the clinical benefit for a patient who is treated with a glycine reuptake inhibitor, and/or
c) for the prediction of the clinical benefit for a patient, having neurodevelopmental, neurological or neuropsychiatric disorders, if treated with a glycine reuptake inhibitor (GRI).

The articles “a” and “an” are used herein to refer to one or to more than one (i.e. to at least one) of the grammatical object of the article. By way of example, “a marker” means one marker or more than one marker. The term “at least” is used to indicate that optionally one or more than one further objects may be present.

The expression “one or more” denotes 1 to 50, preferably 1 to 20 also preferred 2, 3, 4, 5, 6, 7, 8, 9, 10, 12, or 15.

The term “marker” or “biochemical marker” as used herein refers to a molecule to be used as a target for analyzing an individual's test sample. In one embodiment examples of such molecular targets are proteins or polypeptides. Proteins or polypeptides used as a marker in the present invention are contemplated to include naturally occurring variants of said protein as well as fragments of said protein or said variant, in particular, immunologically detectable fragments. Immunologically detectable fragments preferably comprise at least 6, 7, 8, 10, 12, 15 or 20 contiguous amino acids of said marker polypeptide. One of skill in the art would recognize that proteins which are released by cells or present in the extracellular matrix may be damaged, e.g., during inflammation, and could become degraded or cleaved into such fragments. Certain markers are synthesized in an inactive form, which may be subsequently activated by proteolysis. As the skilled artisan will appreciate, proteins or fragments thereof may also be present as part of a complex. Such complex also may be used as a marker in the sense of the present invention. In addition, or in the alternative a marker polypeptide or a variant thereof may carry a post-translational modification. Preferred posttranslational modifications are glycosylation, acylation, or phosphorylation.

A “marker” in the sense of the present invention is a marker that, as single marker, or if combined with the marker CFHR1, adds relevant information in the assessment of a certain disease to the diagnostic question under investigation. The information is considered relevant or of additive value if at a given specificity the sensitivity, or if at a given sensitivity the specificity, respectively, for the assessment of a certain disease can be improved by including said marker into a marker panel (marker combination) already comprising the marker CFHR1. Preferably the improvement in sensitivity or specificity, respectively, is statistically significant at a level of significance of p=0.05, 0.02, 0.01 or lower.

The term “sample” or “test sample” as used herein refers to a biological sample obtained from subject for the purpose of evaluation in vitro. In the methods of the present invention, the sample or patient sample may comprise in an embodiment of the present invention any body fluid. In an embodiment of the present invention, the sample is a body fluid or body liquid, preferably blood, serum, plasma, or liquor. Particularly preferred samples are serum and plasma. The subject is an animal, preferably a human.

Protein concentrations of CFHR1, particularly soluble forms of CFHR1, are determined in vitro in an appropriate sample. According to an embodiment of the present invention “CFHR1” comprises variants or isoforms of CFHR1 according to SEQ ID No. 2, respectively, in particular the variants indicated for SEQ ID No. 2, namely variants H157Y, L159V, E175Q and A296V.

The term “CFH” encompasses all CFH isoforms, including CFH protein isoforms 1 and 2 (denoted CFHL1) according to SEQ ID No. 1.

“About” is understood to mean the indicated value +/−10% standard deviation.

It is known to a person skilled in the art that the detected CFHR1 according to the methods of the present invention will in one embodiment be compared to a reference concentration or amount.

Such reference concentration can be determined using a negative reference sample, a positive reference sample, or a mixed reference sample comprising one or more than one of these types of controls. A negative reference sample preferably will comprise a sample from an apparently healthy individual with no diagnosis of a certain disease or a sample comprising CFHR1 in an amount or concentration corresponding to the amount or concentration of CFHR1 in a sample of an apparently healthy individual with no diagnosis of a certain disease. A positive reference sample preferably will comprise a sample from a subject with the diagnosis of disease or a sample comprising CFHR1 in an amount or concentration corresponding to the amount or concentration of CFHR1 in a sample of a subject with the diagnosis of disease.

The expression “comparing the concentration determined to a reference concentration or amount” is merely used to further illustrate what is obvious to the skilled artisan anyway. A reference concentration is established in a control sample. The control sample may be an internal or an external control sample. In one embodiment an internal control sample is used, i.e. the marker level(s) is (are) assessed in the test sample as well as in one or more other sample(s) taken from the same subject to determine if there are any changes in the level(s) of said marker(s). In another embodiment an external control sample is used. For an external control sample the presence or amount of a marker in a sample derived from the individual is compared to its presence or amount in an individual known to suffer from, or known to be at risk of, a given condition; or an individual known to be free of a given condition, i.e., “normal individual”. For example, a marker concentration in a patient sample can be compared to a concentration known to be associated with a specific course of a certain disease.

Usually the sample's marker concentration is directly or indirectly correlated with a diagnosis and the marker concentration is e.g. used to determine whether an individual is at risk for a certain disease.

Alternatively, the sample's marker concentration can e.g. be compared to a marker concentration known to be associated with a response to therapy in a certain disease, the diagnosis of a certain disease, the assessment of the severity of a certain disease, the guidance for selecting an appropriate drug to a certain disease, in judging the risk of disease progression, or in the follow-up of patients. Depending on the intended diagnostic use an appropriate control sample is chosen and a control or reference value for the marker established therein. It will be appreciated by the skilled artisan that such control sample in one embodiment is obtained from a reference population that is age-matched and free of confounding diseases. As also clear to the skilled artisan, the absolute marker values established in a control sample will be dependent on the assay used. Preferably samples from 100 well-characterized individuals from the appropriate reference population are used to establish a control (reference) value. Also preferred the reference population may be chosen to 30 consist of 20, 30, 50, 200, 500 or 1000 individuals. Healthy individuals represent a preferred reference population for establishing a control value.

The term “measurement”, “measuring” or “determining” preferably comprises a qualitative, a semi-quantitative or a quantitative measurement. In the present invention CFHR1 is preferably measured in a body fluid sample as quantitative measurement, i.e. a distinct concentration of CFHR1 is determined.

The concentration or amount values for CFHR1 as determined in a control group or a control population are in a preferred embodiment used to establish a cut-off value or a reference range. In an embodiment a value above such cut-off value or out-side the reference range at its higher end is considered as elevated or as indicative for the presence of a certain disease or is indicative for the presence of a more severe form of a certain disease. In an embodiment a value below such cut-off value or out-side the reference range at its lower end is considered as lowered or as indicative for the absence of a certain disease or disorder or is indicative for the absence of a more severe form of a certain disease.

In an embodiment of the present invention, a fixed cut-off value is established. Such cut-off value is chosen to match the diagnostic question of interest. In one embodiment, the cut-off is set to result in a specificity of 90%, preferably set to result in a specificity of 95%, more preferably set to result in a specificity of 98%.

In an embodiment the cut-off is set to result in a sensitivity of 90%, also preferred the cut-off is set to result in a sensitivity of 95%, or also preferred the cut-off is set to result in a sensitivity of 98%.

In one embodiment amount or concentration values for CFHR1 determined in a control group or a control population are used to establish a reference range. In a preferred embodiment a concentration or amount of CFHR1 is considered as elevated if the value determined is above the 90%-percentile of the reference range. In further preferred embodiments a protein concentration of CFHR1 is considered as elevated if the value determined is above the 95%-percentile, the 96%-percentile, the 97%-percentile or the 97.5%-percentile of the reference range.

A value above the cut-off value can for example be indicative for the presence of a certain disease, or for a response to a treatment. A value below the cut-off value can for example be indicative for the absence of a certain disease or non-response to a treatment.

As described above, a CFHR1 value above the cut-off value is indicative for a response to GRI treatment of patients having neurodevelopmental, neurological or neuropsychiatric disorders.

In a further preferred embodiment the measurement of CFHR1 is a quantitative measurement. In further embodiments the concentration of CFHR1 is correlated to an underlying diagnostic question.

A sample provided from a patient with already confirmed disease in certain settings might be used as a positive control sample and preferably assayed in parallel with the sample to be investigated. In such setting a positive result for the marker protein CFHR1 in the positive control sample indicates that the testing procedure has worked on the technical level.

As the skilled artisan will appreciate, any such assessment is made in vitro. The sample (test sample) is discarded afterwards. The sample is solely used for the in vitro diagnostic method of the invention and the material of the sample is not transferred back into the patient's body. Typically, the sample is a body fluid sample, e.g., blood, serum, plasma, or liquor. The method according to the present invention is based on a liquid or body fluid sample which is obtained from an individual and on the in vitro determination of protein concentration of CFHR1 in such sample. An “individual”, “subject” or “patient” as used herein refers to a single animal, in particular human.

Preferably the protein concentration of CFHR1 is specifically determined in vitro from a liquid sample by use of a kit of the present invention.

The inventors of the present invention surprisingly are able to detect CFHR1 in a body fluid sample. In a preferred embodiment the method(s) according to the present invention is practiced with serum as sample material. In a further preferred embodiment the method(s) according to the present invention is practiced with plasma as sample material. In a further preferred embodiment the method(s) according to the present invention is practiced with whole blood as sample material. In a further preferred embodiment the method(s) according to the present invention is practiced with liquor as sample material.

In a further embodiment, the present invention relates to use of CFHR1 as a marker molecule in the in vitro assessment of a certain disease from a blood, serum, plasma or liquor sample obtained from an individual, with serum or plasma being preferred.

The ideal scenario for diagnosis would be a situation wherein a single event or process would cause the respective disease as, e.g., in infectious diseases. In all other cases correct diagnosis can be very difficult, especially when the etiology of the disease is not fully understood as is the case for schizophrenia. As the skilled artisan will appreciate, no biochemical marker is diagnostic with 100% specificity and at the same time 100% sensitivity for a given multifactorial disease, as for example for schizophrenia. Rather, biochemical markers are used to assess with a certain likelihood or predictive value an underlying diagnostic question, e.g., the presence, absence, or the severity of a disease. Therefore in routine clinical diagnosis, generally various clinical symptoms and biological markers are considered together in the assessment of an underlying disease. The skilled artisan is fully familiar with the mathematical/statistical methods that routinely are used to calculate a relative risk or likelihood for the diagnostic question to be assessed. In routine clinical practice various clinical symptoms and biological markers are generally considered together by a physician in the diagnosis, treatment, and management of the underlying disease.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons, New York, N.Y. (1994); March, Advanced Organic Chemistry Reactions, Mechanisms and Structure, 4th ed., John Wiley & Sons, New York, N.Y. (1992); Lewin, B., Genes V, published by Oxford University Press (1994), ISBN 0-19-854287-9; Kendrew, J. et al. (eds.), The Encyclopedia of Molecular Biology, published by Blackwell Science Ltd. (1994), ISBN 0-632-02182-9; and Meyers, R. A. (ed.), Molecular Biology and Biotechnology: a Comprehensive Desk Reference, published by VCH Publishers, Inc. (1995), ISBN 1-56081-569-8 provide one skilled in the art with a general guide to many of the terms used in the present application.

The practicing of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, Sambrook et al., Molecular Cloning: A Laboratory Manual, second edition (1989); Gait, M. J., Oligonucleotide Synthesis (1984); Freshney, R. I. (ed.), Animal Cell Culture (1987); Methods in Enzymology, Academic Press, Inc.; Ausubel, F. M. et al. (eds.), Current Protocols in Molecular Biology, (1987) and periodic updates; Mullis et al. (eds.), PCR: The Polymerase Chain Reaction (1994).

### EXAMPLES

### Example 1

### Antibodies Used for the CFHR1-Specific Assays

For the development of a CFHR1-specific assay the following monoclonal antibodies were used: MAB<CFH/CFHR1>M-L20/3 (provider: Thermo Scientific, cat. no.: GAU 020-03-02), MAB<CFHR1>M-442127 (provider: R&D-systems, cat.-no.: MAB4247) and in-house developed monoclonal antibodies MAB<CFHR1>M-4.1.3, MAB<CFHR1>M-4.2.53, MAB<CFHR1>M-4.2.74, MAB<CFHR1>M-5.3.23, and MAB<CFHR1>M-5.1.5, respectively, according to the present invention, which are described in Example 3, 4, 10 and 12.

### Example 2

### Production of Recombinant CFHR1-CFHR5 and CFHL1-Derivatives

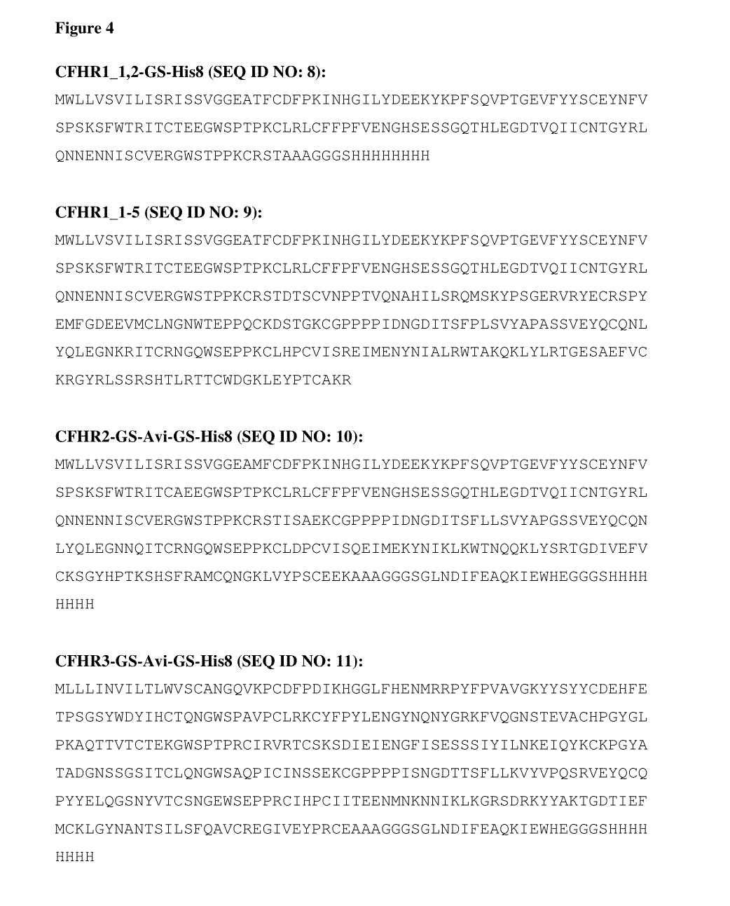

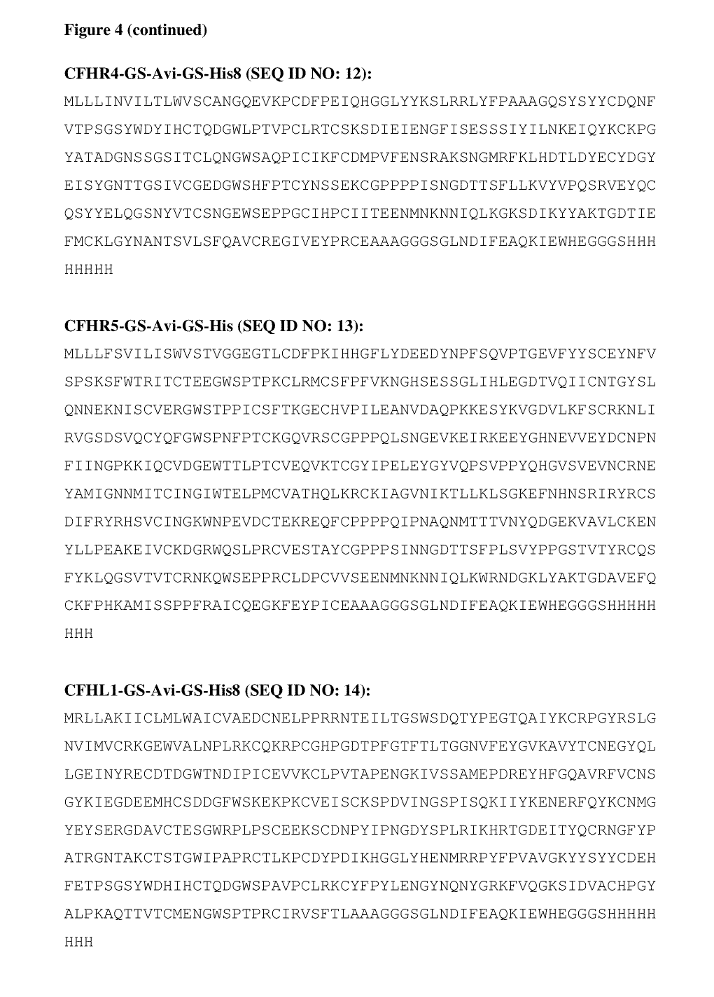

Transient gene expression (TGE) by transfection of plasmid DNA is a rapid strategy to produce proteins in mammalian cell culture. The cDNAs coding for CFHR1, CFHR2, CFHR3, CFHR4B, CFHR5 as well as CFHL1 were purchased from Source BioScience LifeSciences. The coding sequences were PCR amplified and cloned into pM1MT (Roche Applied Science) by standard recombinant cloning techniques into a cassette coding for the Avi-GS-His tag, in order to yield the proteins as listed in FIG. 4, except for CFHR4A. CFHR4A was not produced, since the protein sequence of the SCR-1 of this molecule is identical to the SCR1-2 of CFHR4B and SCR2 of CFHR4A is 92% identical to CFHR4B, therefore the cross-reactivity was assumed to be the same for both CFHR4A and CFHR4B, respectively.

By using pM1MT, expression of the above coding sequences is under control of the human cytomegalovirus (CMV) immediate-early enhancer/promoter region, intron A for enhanced expression and the BGH polyadenylation signal.

Tag-less CFHR1_{—}1-5 corresponding to the full-length mature CFHR1 was generated by introducing a stop codon into CFHR1_{—}1-5-GS-His8 expression construct using QuikChange Site-Directed Mutagenesis (Stratagene) according to the manufacturer's recommendations. All mutations were verified by automated sequencing (Sequiserve).

For transient gene expression in human embryonic kidney (HEK) 293 cells, we used a serum-free and suspension-adapted HEK293 cell line cultured in shaken flasks which was transfected at approx. 1-2×10⁶ vc/ml with the expression plasmid (0.5 to 1 mg/L cell culture) complexed by the 293-Free™ (Merck) transfection reagent. Approx. seven days post-transfection, the culture supernatants were harvested for the downstream process: Following diafiltration against 50 mM K—PO₄ (pH 7.5), 35 mM NaCl (12 mS), addition of Complete protease inhibitor cocktail tablets (Roche) and Benzonase® (Merck) treatment, the His-tagged derivatives were purified by a Ni-NTA (Superflow, Qiagen) chromatography step followed by size exclusion chromatography. Finally, the proteins were dialyzed against 50 mM Hepes pH7.5, 150 mM NaCl, 6.5% Saccharose, 10 mM Cystein and stored at −80° C. resulting in functional and stable proteins fractions of >95% purity as shown by relative titer assay, analytical gelfiltration and/or SDS-PAGE.

### Example 3

### Production of Monoclonal Antibodies MAB<CFHR1>M-5.1.5 MAB<CFHR1>M-4.1.3, MAB<CFHR1>M-4.2.53, MAB<CFHR1>M-4.2.74 and MAB<CFHR1>M-5.3.23

### 3.1. Mice Immunizations

Female BALB/C and/or NMRI mice, respectively, 8-12 weeks old, were immunized three times with recombinant CFHR1_{—}1,2-GS-His8 antigen with the sequence according to SEQ ID No. 8 at three weeks intervals. First injection was performed intraperitoneally with 30 μg antigen emulsified in complete Freund's adjuvant. Second immunization was performed subcutaneously with 10 μg antigen mixed with Abisco adjuvant (Isconova) and third injection occurred intraperitonealy with 5 μg antigen. Ten days after the last immunization blood was taken and the antibody titer was determined in the serum of the immunized mice. Selected mice were given an intravenous booster injection of 50 μg of recombinant CFHR1_{—}1,2_GS_His8 dissolved in PBS three days before fusion.

### 3.2. Hybridoma Production

Spleen cells of the immunized mice were fused with myeloma cells following the procedure of Galfre and Milstein (1981) Meth. Enzymol. 73, 3-46. 1×10⁸ spleen cells of the immunized mouse were mixed with 2×107 myeloma cells (P3X63-Ag8-653, ATCC CRL1580) and centrifuged. The cells were then washed once in RPMI 1640 medium w/o FCS and again centrifuged at 400 g. The supernatant was discarded, the cell sediment was gently loosened by tapping, 1 ml PEG was added to this within one minute and mixed with the cells by gently swirling in a 37° C. warm water bath. Subsequently 5 ml RPMI 1640 medium w/o FCS was added dropwise within 5 min and mixed in a 37° C. warm water bath by continuous swirling. After the addition of 25 ml RPMI 1640 medium w/o FCS the cells were centrifuged for 10 min at 400 g. The cell pellet was taken up in RPMI 1640 medium, 5% FCS and inoculated into azaserine-hypoxanthine selection medium (5.7 μM azaserine, 100 μM hypoxanthine, 2 mM glutamine, 1 mM sodium pyruvate, 50 μM 2-mercaptoethanol and 100 μM non-essential amino acids in RPMI 1640 supplemented with 5% FCS). Mouse recombinant interleukin 6 (50 U/ml) was added to the medium as a growth factor. After 10 days the primary cultures were tested for the synthesis of CFHR1-binding antibodies. CFHR1-binding hybridoma primary cultures were cloned in microtitre plates by means of fluorescence activated cell sorting (FACS).

### 3.3. Determination of the Binding and Specificity of the Produced Antibodies

MAb production in hybridoma culture supernatants was assayed by indirect enzyme-linked immunosorbent assay (ELISA). Streptavidin coated microtiter plates (Microcoat, Bernried, Germany) were incubated with biotinylated Fab fragment of the MAB<CFH/CFHR1>M-L20/3 monoclonal antibody diluted 1:2000 in incubation buffer (phosphate buffered saline pH 7.3, 0.5% Byco C) for 1 h at room temperature. After washing with washing buffer (0.9% NaCl solution, 0.05% Tween 20) the microtiter plates were incubated for 1 h at room temperature with 100 ng/ml purified recombinant CFHR1 and CFH protein diluted in incubation buffer. The microtiter plates were washed again and hybridoma supernatants were added to the coated well and incubated for 1 h at room temperature. After washing, the bound monoclonal antibodies (MAbs) were detected by a 1 h incubation with goat anti-mouse IgG peroxidase conjugate (Calbiochem, Germany) diluted 1:5000 in incubation buffer followed by substrate reaction with ABTS solution (Roche, Germany) after further washing step. The color change was measured in an ELISA reader at 405/490 nm after 20-30 min. Some CFHR1-specific clones without any crossreactivity against CFH were selected.

### 3.4. Production of Sample IgG

Selected hybridoma clones were adapted to serum free medium (HyClone ADCF-MAb; Thermo Fisher) supplemented with 0.1% Nutridoma CS (Roche, Germany) and cultivated in 175 cm² tissue culture flask to a density of 1×10⁵ cells/ml. 2×10⁷ cells obtained from the pre-culture were resuspended in 10 ml of fresh medium and inoculated into the cell compartment of a CELLine classic 1000 bioreactor (Integra Biosciences, Germany). 500 ml of fresh medium were added to the medium compartment and the cells were incubated for 6 to 7 days in CO₂ incubator. After the initial incubation medium change within the medium compartment and harvesting of 5 ml hybridoma suspension from the cell compartment were performed twice a week. The harvested cell suspension was centrifuged at 400 g and the cell free supernatant collected for subsequent IgG purification. One of the clones, namely clone 5.1.5, corresponded to the clone producing MAB<CFHR1>M-5.1.5. Other clones correspond to the clones producing MAB<CFHR1>M-4.1.3, MAB<CFHR1>M-4.2.53, MAB<CFHR1>M-4.2.74, and MAB<CFHR1>M-5.3.23, respectively.

### Example 4

### BiaCore Analysis of the Antibodies MAB<CFH/CFHR1>M-L20/3, MAB<CFHR1>M-442127, MAB<CFHR1>M-4.1.3, and MAB<CFHR1>M-5.1.5

The antibodies MAB<CFH/CFHR1>M-L20/3, MAB<CFHR1>M-442127, MAB<CFHR1>M-4.1.3 and MAB<CFHR1>M-5.1.5 were analyzed on the BiaCore. The chip CM5 coated with a rabbit anti-Mouse IgG was used to bind the purified monoclonal mouse antibodies. In the next step native CFH or the recombinant CFHR1, CFHR2, CFHR3, CFHR4B or CFHR5 proteins according to SEQ ID No.: 9 to 13 were added to determine the binding affinities of the different antibodies. The results are shown in Table 1.

TABLE 1
Binding affinities (KD in nM) of the
different CFHR1 binding antibodies

| | KD[nM] | KD[nM] | KD[nM] | KD[nM] |
|---|---|---|---|---|
| | MAB | MAB | MAB | MAB |
| | <CFH/CFHR1> | <CFHR1> | <CFHR1> | <CFHR1> |
| Antigen | M-L20/3 | M-442127 | M-5.1.5 | M-4.1.3 |
| CFHR1 | 0.1 | 0.1 | 0.01 | 0.8 |
| CFH | 8 | 22 | n.d. | n.d. |
| CFHR2 | n.d. | n.d. | 0.3 | 19 |
| CFHR3 | n.d. | n.d. | n.d. | n.d. |
| CFHR4 | n.d. | n.d. | n.d. | n.d. |
| CFHR5 | 10 | n.d. | n.d. | n.d. |
| CFHL1 | n.d. | n.d. | n.d. | n.d. |

n.d. = not detectable

The antibody MAB<CFH/CFHR1>M-L20/3 shows a high reactivity against CFHR1 and a lower affinity to CFH and CFHR5. Surprisingly, the affinity against CFHR1 is higher than the reactivity against CFH, despite this antibody was generated by immunization with CFH. The three CFHR1-binding antibodies MAB<CFHR1>M-442127, MAB<CFHR1>M-5.1.5, and MAB<CFHR1>M-4.1.3 show a different reaction pattern. The antibodies MAB<CFHR1>M-5.1.5 and MAB<CFHR1>M-4.1.3 reveal a high reactivity with CFHR1 and only a small reactivity against CFHR2 without any measurable binding of CFH and CFHR5, respectively. In contrary the antibody MAB<CFHR1>M-442127 shows no crossreactivity against CFHR2 and CFHR5, but a low binding of CFH. Due to the crossreactivity to CFH this antibody is less suitable to develop a specific CFHR1 assay. Since MAB<CFHR1>M-5.1.5 and MAB<CFHR1>M-4.1.3 have no crossreactivity against CFH and no crossreactivity against CFHR5, a CFHR1-specific assay is possible in combination with MAB<CFH/CFHR1>M-L20/3.

Thus, similar results were observed for MAB<CFHR1>M-4.1.3 regarding the crossreactivity profile compared to MAB<CFHR1>M-5.1.5. The affinity of MAB<CFHR1>M-4.1.3 to CFHR1 is however lower compared to MAB<CFHR1>M-5.1.5. Therefore, also the MAB<CFHR1>M-4.1.3 antibody may be used in combination with MAB<CFH/CFHR1>M-L20/3 in kits, assays and methods of the present invention.

### Example 5

### Biotinylation of Fab-Fragments of Monoclonal MAB<CFH/CFHR1>M-L20/3; Stoichiometry 1:1.3

Monoclonal mouse IgG of clone L20/3 (provider: Thermo Scientific, cat. no.: GAU 020-03-02) were digested using Papain (3 mU/mg IgG) to produce Fab-fragments. Digested Fc-fragments were eliminated by chromatography on DAE-Sepharose. Purification of Fab by Fcγ-adsorption of remaining Fc followed by Superdex 200 size exclusion.

To a solution of 10 mg/ml L20/3-Fab fragments in 100 mM KPO₄, pH 8.5 50 ul Biotin-N-hydroxysuccin-imide (3.6 mg/ml in DMSO) were added per ml. After 45 min at room temperature, the sample was dialysed against 100 mM KPO₄, 150 mM NaCl, pH 7.2 and frozen.

### Example 6

### Ruthenylation of Monoclonal MAB<CFHR1>M-442127 and all in-House Monoclonal MABs<CFHR1>M i.e. 5.1.5, 4.1.3., 4.2.53, 4.2.74 and 5.3.23, Respectively; Stoichiometry 1:3

To a solution of 5 mg/ml monoclonal mouse IgG (MAB<CFHR1>M-442127; clone 442127, provider: R&D-systems, cat.-no.: MAB4247 or the in-house MABs<CFHR1> as listed in the heading, respectively, in 100 mM KPO4, pH 8.5, 125 ug Ruthenium-(bpy)2-bpyCO-Osu were added. After 75 min at room temperature, ruthenylation was stopped by addition of 10 mM Lysine. For separation of aggregates appropriate fractions of sample were collected from Superdex 200 size exclusion chromatography.

### Example 7

### CFHR1-Assay 1 Using Biotinylated L20/3 Fab Fragment and Ruthenylated MAB<CFHR1>M-5.1.5-Ru

An electrochemiluminescence immunoassay (ECLIA) for the specific measurement of CFHR1 in particular in human serum or plasma samples was developed using the Elecsys® cobas analyzer e601. The Elecsys CFHR1 immunoassay is an electrochemiluminescence immunoassay (ECLIA) that functions via the sandwich principle. There are two antibodies included in the assay, namely a biotinylated Fab fragment of monoclonal antibody MAB<CFH/CFHR1>M-L20/3 (L20/3-Bi; capture antibody) and a ruthenylated monoclonal anti-CFHR1 antibody MAB<CFHR1>M-5.1.5 (MAB<CFHR1>M-5.1.5-Ru; detection antibody), which form sandwich immunoassay complexes with CFHR1 in the sample. The complexes are then bound to solid-phase streptavidin-coated microparticles. The microparticles are magnetically captured onto the surface of an electrode, and the application of a voltage to the electrode induces chemiluminescent emission, which is measured by a photomultiplier for readouts. Results are determined via an instrument-specific calibration curve.

Samples are diluted 1:400 using the Diluent Universal (Roche Diagnostics GmbH, No. 03183971). Assay protocol 10 is applied allowing 9 min pre-incubation of 10 ul of the pre-diluted sample with 80 ul of reagent 1 (R1) containing 1.5 μg/ml of biotinylated MAB<CFH/CFHR1>M-L20/3 Fab fragment in reaction buffer (Hepes 50 mM, NaCl 150 mM; Thesit/Polidocanol 0.1%; EDTA 1 mM; bovine serum albumin 0.5%) and reagent 2 (R2) containing 1.0 μg/ml ruthenylated MAB<CFHR1>M-5.1.5 in the same reaction buffer. In the second step 30 μl of a microparticle suspension is added and incubated for further 9 min. During incubation an antibody-analyte-antibody sandwich is formed that is bound to the microparticles. Finally the microparticles are transferred to the detection chamber of the Elecsys system for signal generation and readout. For calibration a series of calibrators with different concentrations of recombinant CFHR1 (0 ng/ml, 7.5 ng/ml, 15.25 ng/ml, 30.25 ng/ml, 60.75 ng/ml and 121.2 ng/ml) are prepared in diluent Universal (Roche-Id. 03609987 190). The equation of the calibration curve was calculated by non-linear least-squares curve-fitting (RCM-Rodbard) and used for converting the signal readout into the corresponding concentration value. The results were multiplied by the dilution factor of the assay (=400).

### Example 8

### Description of Other CFHR1-Assays Using Biotinylated L20/3 Fab Fragment and MAB<CFHR1>M-442127-Ru, MAB<CFHR1>M-4.1.3-Ru, MAB<CFHR1>M-4.2.53-Ru, MAB<CFHR1>M-4.2.74-Ru and MAB<CFHR1>M-5.3.23-Ru, Respectively

For these different sandwich assays the same biotinylated Fab fragment of monoclonal antibody Fab L20/3-Bi (capture antibody) was used in the same buffer composition as in Example 7. The difference regarding to CFHR1 assay 1 was the use of the ruthenylated (=-Ru) monoclonal anti-CFHR1 antibodies MAB<CFHR1>M-442127-Ru, MAB<CFHR1>M-4.1.3-Ru, MAB<CFHR1>M-4.2.53-Ru, MAB<CFHR1>M-4.2.74-Ru and MAB<CFHR1>M-5.3.23-Ru, respectively. These antibodies were also used in the reagent 2 ((R2), Hepes 50 mM, NaCl 150 mM; Thesit/Polidocanol 0.1%; EDTA 1 mM; bovine serum albumin 0.5%) at a concentration of 0.5 mg/L, 2 mg/L, 2 mg/L, 1.5 mg/L and 2 mg/L respectively. The assay procedure and the calibration was according to CFHR1-Assay 1 (see Example 7).

### Example 9

### Crossreactivity of in-House CFHR1 Assays to CFH, CFHR2 and CFHR5

For all 5 in-house CFHR1 assays of Examples 7 and 8 the crossreactivity against the most critical CFH-related proteins was determined by measuring distinct concentrations of the potentially crossreacting serum components CFH (in-house purified native CFH), and the CFHR2 and CFHR5 proteins produced recombinantly. For this experiment the potentially crossreacting proteins were dissolved in the assay diluent and measured according to the assay description. The results are shown in Table 2.

TABLE 2
Analysis of potentially crossreacting proteins

| | | Assay | | | | | |
|---|---|---|---|---|---|---|---|
| | | | | Assay 2 | | Assay 4 | |
| | | | | L20/3- | Assay 3 | L20/3- | Assay 3 |
| | | Assay 1 | Assay 1 | Bi/ | L20/3-Bi/ | Bi/ | L20/3-Bi/ |
| | | L20/3-Bi/ | L20/3- | 4.1.3- | 4.2.53- | 4.2.74- | 5.3.23- |
| | Weighted | 5.1.5-Ru | Bi/ | Ru | Ru | Ru | Ru |
| | concentration | Measured | 5.1.5- | Measured | Measured | Measured | Measured |
| | of | CFHR1 | Ru | CFHR1 | CFHR1 | CFHR1 | CFHR1 |
| | crossreactant | [μg/ml] | X-R [%] | [μg/ml] | [μg/ml] | [μg/ml] | [μg/ml] |
| CFH | 500 μg/ml | 0.5 | 0.1 | 0.5 | 0.5 | 0.5 | 0.5 |
| (in- | 2000 μg/ml | 1.9 | 0.1 | 2.0 | 1.9 | 2.1 | 2.1 |
| house) | | | | | | | |
| CFHR2 | 50 μg/ml | 0.0 | 0.0 | 0.0 | 0.0 | 0.0 | 0.0 |
| | 100 μg/ml | 0.0 | 0.0 | 0.0 | 0.09 | 0.0 | 0.0 |
| CFHR5 | 25 μg/ml | 0.0 | 0.0 | 0.0 | 0.0 | 0.0 | 0.0 |
| | 50 μg/ml | 0.0 | 0.0 | 0.0 | 0.0 | 0.0 | 0.0 |

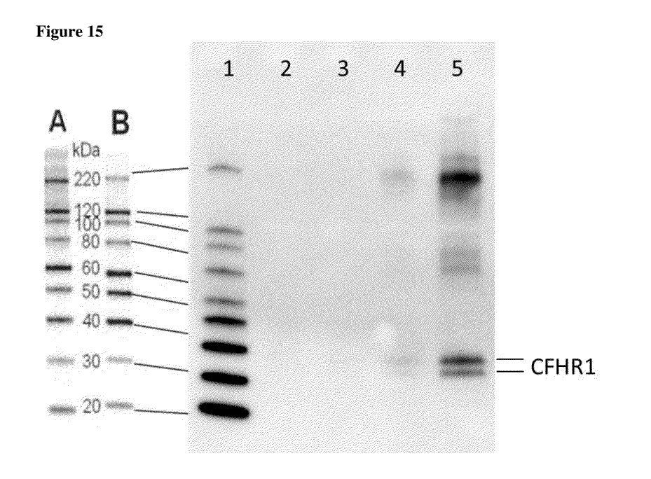

All 5 assays show identical crossreactivity. There is no cross-reactivity against CFHR2 and CFHR5 and a very low reactivity of 0.1% against CFH. Due to the fact, that the purified CFH show a impurity of CFHR1 (see Western blot analysis of FIG. 15), the true (cross-)reactivity, if any, against CFH is lower than 0.1%.

### Example 10

### Crossreactivity of Two Selected CFHR1 Assays to all Other CFH-Related Proteins

The (cross-)reactivity of the preferred CFHR1 assay (L20/3-Bi/5.1.5-Ru) and the CFHR assay using the MAK<CFHR1>M-442127 of Examples 7 and 8, respectively, was determined by measuring distinct concentrations of the potentially crossreacting serum components CFH (purified native CFH, No. 4400-9554, AbDSerotec) and the tagged recombinant proteins CFHL1, CFHR2, CFHR3, CFHR4B and CFHR5. For this experiment purified CFH from AbDSerotec was used, since this material shows very low contamination of CFHR1. The potentially crossreacting proteins were dissolved in the assay diluent and measured according to the assay description. These results are shown in Table 3. In addition a second experiment was carried out to specifically check the crossreactivity against CFH. Samples collected from patients with a CFHR1 deletion; i.e. samples in which no CFHR1 is present, were selected and measured in both assays. The results are shown in Table 4 further below.

TABLE 3
Analysis of potentially crossreacting proteins

| | | Assay 1 | | | |
|---|---|---|---|---|---|
| | | L20/3-Bi/MAB<CFHR1> | Assay 1 | Assay 2 | |
| | | M-5.1.5-Ru | L20/3-Bi/MAB<CFHR1> | L20/3-Bi/442127-Ru | Assay 2 |
| | Weighted | Measured CFHR1 | M-5.1.5-Ru | Measured CFHR1 | L20/3-Bi/442127-Ru |
| | concentration | concentration | Cross-reactivity | concentration | Cross-reactivity |
| Assay | of cross-reactant | [μg/ml] | [%] | [μg/ml] | [%] |
| CFH | 125 μg/ml | 0.017 | 0.014 | 0.97 | 0.78 |
| | 250 μg/ml | 0.036 | 0.014 | 1.92 | 0.77 |
| | 500 μg/ml | 0.074 | 0.015 | 4.01 | 0.80 |
| | 1000 μg/ml | 0.158 | 0.016 | 9.71 | 0.97 |
| CFHR2 | 50 μg/ml | 0.0 | 0.0 | 0.0 | 0.0 |
| | 100 μg/ml | 0.003 | 0.0 | 0.0 | 0.0 |
| CFHR3 | 21 μg/ml | 0.0 | 0.0 | 0.0 | 0.0 |
| | 50 μg/ml | 0.0 | 0.0 | 0.0 | 0.0 |
| CFHR4 | 21 μg/ml | 0.0 | 0.0 | 0.0 | 0.0 |
| | 50 μg/ml | 0.0 | 0.0 | 0.0 | 0.0 |
| CFHR5 | 21 μg/ml | 0.0 | 0.0 | 0.0 | 0.0 |
| | 50 μg/ml | 0.0 | 0.0 | 0.0 | 0.0 |
| CFHL1 | 50 μg/ml | 0.0 | 0.0 | 0.0 | 0.0 |
| | 100 μg/ml | 0.0 | 0.0 | 0.001 | 0.0 |

For the specific in-house CFHR1 assay (Example 7) using the monoclonal antibody MAB<CFHR1>M-5.1.5 of the present invention, no significant crossreactivity has been determined for CFH and no cross-reactivity against all other CFH-related proteins CFHL1, CFHR2, CFHR3, CFHR4B, CFHR5. The limit of quantification of assay 1 was assessed as <0.25 μg/ml and the measured CFHR1-concentration for CFH as crossreactant is below the measuring range. On the other hand the CFHR1 assay 2 using the CFHR1-specific antibody MAB<CFHR1>M-442127 shows some crossreactivity against CFH with approx. 0.8-1.0%. This is a critical value, since CFH concentrations in serum are significantly higher than the CFHR1 concentrations.

Thus, specific assays for CFHR1 were established.

TABLE 4
Results of human serum samples collected
from patients with a CFHR1 deletion

| | Assay 1 | |
|---|---|---|
| | L20/3-Bi/MAB<CFHR1> | Assay 2 |
| | M-5.1.5-Ru | L20/3-Bi/442127-Ru |
| Assay | Measured CFHR1 | Measured CFHR1 |
| Sample | concentration | concentration |
| number | [μg/ml] | [μg/ml] |
| 1 | 0.096 | 2.97 |
| 2 | 0.024 | 2.92 |
| 3 | 0.038 | 2.88 |
| 4 | 0.041 | 2.36 |
| 5 | 0.028 | 2.64 |
| 6 | 0.022 | 3.36 |

### Example 11

### Production of CFHR1 Reference Material

Recombinant CFHR1_{—}1-5 with a sequence according to SEQ ID No. 9 (AA19-330, no affinity tag) was transiently expressed in human embryonic kidney (HEK) 293 cells. The cleared cell culture supernatant was further purified by immunoaffinity chromatography using a monoclonal antibody specific for CFHR1 immobilized on a column matrix. The affinity column was loaded with the recombinant CFHR1 and washed with 10 mM Tris/HCl, 20 mM NaCl pH 8.5 and 10 mM Tris/HCl, 500 mM NaCl, 0.05% Tween 20 pH 8.5 to remove non-specifically bound proteins. RecCFHR1 was eluted from the column with 1M propionic acid and the pH of the eluate was adjusted to 8.5 using 2M Arginine/HCl pH 9.2. Following dialysis against 5 mM potassium phosphate, 5 mM NaCl pH 8.5 the affinity purified recCFHR1 was captured on an ion exchange chromatography column (Resource Q, GE Health Care Life Sciences) and eluted in a NaCl gradient. The product was dialysed against a storage buffer (50 mM potassium phosphate, 150 mM NaCl pH 8.5), cleared by filtration using a 0.2 μm Supor® PES membrane disc filter (Pall Corporation) and stored frozen at −80° C.

### Example 12

### Sequence Analysis of MAB<CFHR1>M-5.1.5, MAB<CFHR1>M-4.1.3, MAB<CFHR1>M-4.2.53, MAB<CFHR1>M-4.2.74, and MAB<CFHR1>M-5.3.23

Sequence analysis of mouse monoclonal antibody <CFHR1>M-5.1.5 from the clone identified in Examples 3 and 4 was performed by RACE-PCR according to Doeneke et al. (1997) Leukemia 11, 1787-1792 using the 5′/3′ RACE Kit, 2^{nd} Generation (Roche Applied Science). The results of the sequence analysis are shown in FIGS. 2 and 3. The sequences are shown in SEQ ID No. 15 to 18.

Sequence analysis of mouse monoclonal antibodies MAB<CFHR1>M-4.1.3, MAB<CFHR1>M-4.2.53, MAB<CFHR1>M-4.2.74, and MAB<CFHR1>M-5.3.23 identified in Example 3 was performed accordingly. The results of the sequence analyses are shown in FIGS. 5 to 12. The amino acid and DNA sequences are shown in SEQ ID No. 24 to 39.

All references cited in this specification are herewith incorporated by reference with respect to their entire disclosure content and the disclosure content specifically mentioned in this specification.

While this disclosure has been described using certain examples and disclosures, the present disclosure may be further modified within the spirit and scope of this disclosure. This application is therefore intended to cover any variations, uses, or adaptations of the disclosure using its general principles. Further, this application is intended to cover such departures from the present disclosure as come within the known or customary practice in the art to which this disclosure pertains.

1. A kit comprising
a) a first agent capable of binding complement factor H R1 (CFHR1) protein, and
b) a second agent capable of binding complement factor H R1 (CFHR1) protein,
wherein the first agent and the second agent bind to different and non-overlapping epitopes,
and wherein the first agent and the second agent do not both crossreact with CFH,
and wherein the first agent and the second agent do not both crossreact with CFHR2,
and wherein the first agent and the second agent do not both crossreact with CFHR3,
and wherein the first agent and the second agent do not both crossreact with CFHR4,
and wherein the first agent and the second agent do not both crossreact with CFHR5,
and wherein one agent is labeled with a detectable label,
and wherein the other agent is capable of immobilizing on a solid phase.

2. The kit according to claim 1,
a) wherein the first agent binds to an epitope within amino acids 144-330 of SEQ ID No. 2, and/or
b) wherein the second agent binds to an epitope within amino acids 1-143 of SEQ ID No. 2.

3. The kit according to claim 1, wherein
a) the first agent crossreacts with CFH and CFHR5, but does not crossreact with CFHR2, CFHR3, and CFHR4, and/or
b) wherein the second agent crossreacts with CFHR2, but does not crossreact with CFH, CFHR3, CFHR4 and CFHR5.

4. The kit according to claim 1, wherein
a) the first agent is an antibody, in particular the antibody MAB<CFH/CFHR1>M-L20/3, wherein the heavy chain has a sequence of SEQ ID No. 41, and wherein the light chain has a sequence of SEQ ID No. 43 or an antibody comprising the CDR sequences of MAB<CFH/CFHR1>M-L20/3, and/or
b) the second agent is an antibody, in particular the antibody MAB<CFHR1>M-5.1.5, wherein the heavy chain has a sequence of SEQ ID No. 16, and wherein the light chain has a sequence of SEQ ID No. 18, or antibody comprising the CDR sequences of MAB<CFHR1>M-5.1.5 according to SEQ ID No. 19, SEQ ID No. 20, SEQ ID No. 21, SEQ ID No. 22, and SEQ ID No. 23 and the amino acid sequence ITS.

5. A method for an in vitro assay for detecting complement factor H R1 (CFHR1) protein in a sample obtained from a subject, comprising
a) contacting the sample with the agents of any of the kits of claim 1,
b) immobilizing the formed complexes to a solid phase, and
c) detecting CFHR1, in particular determining the amount and/or concentration of CFHR1,
wherein step b) may be performed before step a), after step a) or simultaneously with step a).

6. An in vitro method of predicting a response for patients, having neurodevelopmental, neurological or neuropsychiatric disorders, if treated with a glycine reuptake inhibitor (GRI), comprising the steps
i) determining the protein concentration of CFHR1 in a sample of a patient by performing the assay of claim 5,
ii) comparing the protein concentration determined in step i) to a cut-off value for CFHR1 in patients, having neurodevelopmental, neurological or neuropsychiatric disorders,
iii) wherein a protein concentration CFHR1 in the sample of the patient having neurodevelopmental, neurological or neuropsychiatric disorders above the cut-off value is indicative for a patient who will derive clinical benefit from treatment GRI, and
iv) selecting a GRI treatment for patients having neurodevelopmental, neurological or neuropsychiatric disorders.

7. The method according to claim 5, wherein said sample is blood, serum, liquor or plasma.

8. The method according to claim 6,
a) wherein the neurodevelopmental, neurological or neuropsychiatric disorders include negative or positive symptoms of schizophrenia, bipolar disorder, substance dependence, autism and compulsive disorders, in particular negative or positive symptoms of schizophrenia, and/or
b) wherein the patient is affected with schizoaffective disorder; and/or
c) wherein the GRI is [4-(3-fluoro-5-trifluormethyl-pyridin-2-yl)-piperazin-1-yl]-[5-methanesulfonyl-2-[[(2S)-1,1,1-trifluoropropan-2-yl]oxy]phenyl]methanone.

9. The method according to claim 5,
a) wherein the assay is an enzyme-linked immunoassay (ELISA) or electrochemiluminescence immunoassay (ECLIA) or radioimmunoassay (RIA), and/or
b) wherein the detection range of CFHR1 protein is 0.02 to about 50 μg/ml, more preferred from about 0.05 to about 35 μg/ml, and/or
c) wherein the first agent and second agent are monoclonal antibodies, and/or
d) wherein the first agent is MAB<CFH/CFHR1>M-L20/3, which is labeled with a detectable label, and the second agent is MAB<CFHR1>M-5.1.5, which is capable of immobilizing on a solid phase, and/or
e) wherein the assay is standardized by a recombinant CFHR1 protein calibrator, in particular wherein the recombinant CFHR1 protein is produced in HEK cells,
f) wherein the agent with less cross-reactivity to CFH, CFHR2, CFHR3, CFHR4 and/or CFHR5 is contacted with the sample according to step a) of claim 6 before contacting the other agent with the sample.

10. The method according to claim 5,
a) wherein the first agent is capable of immobilizing on a solid phase and the second agent is labeled with a detectable label, or
b) wherein the first agent is labeled with a detectable label and the second agent is capable of immobilizing on a solid phase on a solid phase.

11. An agent capable of binding CFHR1, which agent is
a) (i) monoclonal antibody MAB<CFHR1>M-5.1.5, wherein the heavy chain has a sequence of SEQ ID No. 16, and wherein the light chain has a sequence of SEQ ID No. 18, or
(ii) monoclonal antibody MAB<CFHR1>M-4.1.3, wherein the heavy chain has a sequence of SEQ ID No. 25, and wherein the light chain has a sequence of SEQ ID No. 27, or
(iii) monoclonal antibody MAB<CFHR1>M-4.2.53, wherein the heavy chain has a sequence of SEQ ID No. 29, and wherein the light chain has a sequence of SEQ ID No. 31, or
(iv) monoclonal antibody MAB<CFHR1>M-4.2.74, wherein the heavy chain has a sequence of SEQ ID No. 33, and wherein the light chain has a sequence of SEQ ID No. 35, or
(v) monoclonal antibody MAB<CFHR1>M-5.3.23, wherein the heavy chain has a sequence of SEQ ID No. 37, and wherein the light chain has a sequence of SEQ ID No. 39,
or
b) (i) an antibody comprising the CDR sequences of MAB<CFHR1>M-5.1.5 according to SEQ ID No. 19, SEQ ID No. 20, SEQ ID No. 21, SEQ ID No. 22, and SEQ ID No. 23 and the amino acid sequence ITS and/or comprising the CDR3 sequences of MAB<CFHR1>M-5.1.5 according to SEQ ID No. 21 and SEQ ID No. 23, or
(ii) an antibody comprising the CDR sequences of MAB<CFHR1>M-4.1.3, MAB<CFHR1>M-4.2.53, MAB<CFHR1>M-4.2.74, or MAB<CFHR1>M-5.3.23 and/or comprising the CDR3 sequences of MAB<CFHR1>M-4.1.3, MAB<CFHR1>M-4.2.53, MAB<CFHR1>M-4.2.74, or MAB<CFHR1>M-5.3.23,
or
c) a functionally active variant of a) or b).

12. One or more nucleic acid(s) coding for the antibody according to claim 11, wherein the nucleic acid(s) is/are located in a vector,
and/or
(i) the nucleic acids according to SEQ ID No. 15 and 17, and/or
(ii) the nucleic acids according to SEQ ID No. 24 and 26, and/or
(iii) the nucleic acids according to SEQ ID No. 28 and 30, and/or
(iv) the nucleic acids according to SEQ ID No. 32 and 34, and/or
(v) the nucleic acids according to SEQ ID No. 36 and 38,
wherein the nucleic acid(s) is/are located in a vector.

13. A cell line producing an agent which agent is a:
a) (i) monoclonal antibody MAB<CFHR1>M-5.1.5, wherein the heavy chain has a sequence of SEQ ID No. 16, and wherein the light chain has a sequence of SEQ ID No. 18, or
(ii) monoclonal antibody MAB<CFHR1>M-4.1.3, wherein the heavy chain has a sequence of SEQ ID No. 25, and wherein the light chain has a sequence of SEQ ID No. 27, or
(iii) monoclonal antibody MAB<CFHR1>M-4.2.53, wherein the heavy chain has a sequence of SEQ ID No. 29, and wherein the light chain has a sequence of SEQ ID No. 31, or
(iv) monoclonal antibody MAB<CFHR1>M-4.2.74, wherein the heavy chain has a sequence of SEQ ID No. 33, and wherein the light chain has a sequence of SEQ ID No. 35, or
(v) monoclonal antibody MAB<CFHR1>M-5.3.23, wherein the heavy chain has a sequence of SEQ ID No. 37, and wherein the light chain has a sequence of SEQ ID No. 39,
or
b) (i) an antibody comprising the CDR sequences of MAB<CFHR1>M-5.1.5 according to SEQ ID No. 19, SEQ ID No. 20, SEQ ID No. 21, SEQ ID No. 22, and SEQ ID No. 23 and the amino acid sequence ITS and/or comprising the CDR3 sequences of MAB<CFHR1>M-5.1.5 according to SEQ ID No. 21 and SEQ ID No. 23, or
(ii) an antibody comprising the CDR sequences of MAB<CFHR1>M-4.1.3, MAB<CFHR1>M-4.2.53, MAB<CFHR1>M-4.2.74, or MAB<CFHR1>M-5.3.23 and/or comprising the CDR3 sequences of MAB<CFHR1>M-4.1.3, MAB<CFHR1>M-4.2.53, MAB<CFHR1>M-4.2.74, or MAB<CFHR1>M-5.3.23,
or
c) a functionally active variant of a) or b).

14. The agent capable of binding CFHR1 of claim 11,
labeled with a detectable label, and/or capable of immobilizing on a solid phase.


SEQUENCE LISTING (43 sequences)

SEQ ID NO: 1 - protein, 1231 aa, Mus musculus
  MISC_FEATURE (1)..(1231): human complement factor H (CFH) and isoform 2 (CFHL1)
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 2 - protein, 330 aa, Mus musculus
  MISC_FEATURE (1)..(330): human complement factor H related protein 1 (CFHR1)
MWLLVSVILISRISSVGGEATFCDFPKINHGILYDEEKYKPFSQVPTGEVFYYSCEYNFVSPSKSFWTRITCTEEGWSPTPKCLRLCFFPFVENGHSESSGQTHLEGDTVQIICNTGYRLQNNENNISCVERGWSTPPKCRSTDTSCVNPPTVQNAHILSRQMSKYPSGERVRYECRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYLRTGESAEFVCKRGYRLSSRSHTLRTTCWDGKLEYPTCAKR

SEQ ID NO: 3 - protein, 270 aa, Mus musculus
  MISC_FEATURE (1)..(270): human complement factor H related protein 2 (CFHR2)
MWLLVSVILISRISSVGGEAMFCDFPKINHGILYDEEKYKPFSQVPTGEVFYYSCEYNFVSPSKSFWTRITCAEEGWSPTPKCLRLCFFPFVENGHSESSGQTHLEGDTVQIICNTGYRLQNNENNISCVERGWSTPPKCRSTISAEKCGPPPPIDNGDITSFLLSVYAPGSSVEYQCQNLYQLEGNNQITCRNGQWSEPPKCLDPCVISQEIMEKYNIKLKWTNQQKLYSRTGDIVEFVCKSGYHPTKSHSFRAMCQNGKLVYPSCEEK

SEQ ID NO: 4 - protein, 330 aa, Mus musculus
  MISC_FEATURE (1)..(330): human complement factor H related protein 3 (CFHR3)
MLLLINVILTLWVSCANGQVKPCDFPDIKHGGLFHENMRRPYFPVAVGKYYSYYCDEHFETPSGSYWDYIHCTQNGWSPAVPCLRKCYFPYLENGYNQNYGRKFVQGNSTEVACHPGYGLPKAQTTVTCTEKGWSPTPRCIRVRTCSKSDIEIENGFISESSSIYILNKEIQYKCKPGYATADGNSSGSITCLQNGWSAQPICINSSEKCGPPPPISNGDTTSFLLKVYVPQSRVEYQCQPYYELQGSNYVTCSNGEWSEPPRCIHPCIITEENMNKNNIKLKGRSDRKYYAKTGDTIEFMCKLGYNANTSILSFQAVCREGIVEYPRCE

SEQ ID NO: 5 - protein, 578 aa, Mus musculus [sequence omitted]

SEQ ID NO: 6 - protein, 331 aa, Mus musculus
  MISC_FEATURE (1)..(331): human complement factor H related protein 4B (CFHR4B)
MLLLINVILTLWVSCANGQEVKPCDFPEIQHGGLYYKSLRRLYFPAAAGQSYSYYCDQNFVTPSGSYWDYIHCTQDGWSPTVPCLRTCSKSDIEIENGFISESSSIYILNKEIQYKCKPGYATADGNSSGSITCLQNGWSAQPICIKFCDMPVFENSRAKSNGMRFKLHDTLDYECYDGYEISYGNTTGSIVCGEDGWSHFPTCYNSSEKCGPPPPISNGDTTSFLLKVYVPQSRVEYQCQSYYELQGSNYVTCSNGEWSEPPRCIHPCIITEENMNKNNIQLKGKSDIKYYAKTGDTIEFMCKLGYNANTSVLSFQAVCREGIVEYPRCE

SEQ ID NO: 7 - protein, 569 aa, Mus musculus
  MISC_FEATURE (1)..(569): human complement factor H related protein 5 (CFHR5)
MLLLFSVILISWVSTVGGEGTLCDFPKIHHGFLYDEEDYNPFSQVPTGEVFYYSCEYNFVSPSKSFWTRITCTEEGWSPTPKCLRMCSFPFVKNGHSESSGLIHLEGDTVQIICNTGYSLQNNEKNISCVERGWSTPPICSFTKGECHVPILEANVDAQPKKESYKVGDVLKFSCRKNLIRVGSDSVQCYQFGWSPNFPTCKGQVRSCGPPPQLSNGEVKEIRKEEYGHNEVVEYDCNPNFIINGPKKIQCVDGEWTTLPTCVEQVKTCGYIPELEYGYVQPSVPPYQHGVSVEVNCRNEYAMIGNNMITCINGIWTELPMCVATHQLKRCKIAGVNIKTLLKLSGKEFNHNSRIRYRCSDIFRYRHSVCINGKWNPEVDCTEKREQFCPPPPQIPNAQNMTTTVNYQDGEKVAVLCKENYLLPEAKEIVCKDGRWQSLPRCVESTAYCGPPPSINNGDTTSFPLSVYPPGSTVTYRCQSFYKLQGSVTVTCRNKQWSEPPRCLDPCVVSEENMNKNNIQLKWRNDGKLYAKTGDAVEFQCKFPHKAMISSPPFRAICQEGKFEYPICE

SEQ ID NO: 8 - protein, 158 aa, Mus musculus
MWLLVSVILISRISSVGGEATFCDFPKINHGILYDEEKYKPFSQVPTGEVFYYSCEYNFVSPSKSFWTRITCTEEGWSPTPKCLRLCFFPFVENGHSESSGQTHLEGDTVQIICNTGYRLQNNENNISCVERGWSTPPKCRSTAAAGGGSHHHHHHHH

SEQ ID NO: 9 - protein, 330 aa, Mus musculus; identical to SEQ ID NO: 2

SEQ ID NO: 10 - protein, 304 aa, Mus musculus
MWLLVSVILISRISSVGGEAMFCDFPKINHGILYDEEKYKPFSQVPTGEVFYYSCEYNFVSPSKSFWTRITCAEEGWSPTPKCLRLCFFPFVENGHSESSGQTHLEGDTVQIICNTGYRLQNNENNISCVERGWSTPPKCRSTISAEKCGPPPPIDNGDITSFLLSVYAPGSSVEYQCQNLYQLEGNNQITCRNGQWSEPPKCLDPCVISQEIMEKYNIKLKWTNQQKLYSRTGDIVEFVCKSGYHPTKSHSFRAMCQNGKLVYPSCEEKAAAGGGSGLNDIFEAQKIEWHEGGGSHHHHHHHH

SEQ ID NO: 11 - protein, 364 aa, Mus musculus
MLLLINVILTLWVSCANGQVKPCDFPDIKHGGLFHENMRRPYFPVAVGKYYSYYCDEHFETPSGSYWDYIHCTQNGWSPAVPCLRKCYFPYLENGYNQNYGRKFVQGNSTEVACHPGYGLPKAQTTVTCTEKGWSPTPRCIRVRTCSKSDIEIENGFISESSSIYILNKEIQYKCKPGYATADGNSSGSITCLQNGWSAQPICINSSEKCGPPPPISNGDTTSFLLKVYVPQSRVEYQCQPYYELQGSNYVTCSNGEWSEPPRCIHPCIITEENMNKNNIKLKGRSDRKYYAKTGDTIEFMCKLGYNANTSILSFQAVCREGIVEYPRCEAAAGGGSGLNDIFEAQKIEWHEGGGSHHHHHHHH

SEQ ID NO: 12 - protein, 365 aa, Mus musculus
MLLLINVILTLWVSCANGQEVKPCDFPEIQHGGLYYKSLRRLYFPAAAGQSYSYYCDQNFVTPSGSYWDYIHCTQDGWLPTVPCLRTCSKSDIEIENGFISESSSIYILNKEIQYKCKPGYATADGNSSGSITCLQNGWSAQPICIKFCDMPVFENSRAKSNGMRFKLHDTLDYECYDGYEISYGNTTGSIVCGEDGWSHFPTCYNSSEKCGPPPPISNGDTTSFLLKVYVPQSRVEYQCQSYYELQGSNYVTCSNGEWSEPPGCIHPCIITEENMNKNNIQLKGKSDIKYYAKTGDTIEFMCKLGYNANTSVLSFQAVCREGIVEYPRCEAAAGGGSGLNDIFEAQKIEWHEGGGSHHHHHHHH

SEQ ID NO: 13 - protein, 603 aa, Mus musculus
MLLLFSVILISWVSTVGGEGTLCDFPKIHHGFLYDEEDYNPFSQVPTGEVFYYSCEYNFVSPSKSFWTRITCTEEGWSPTPKCLRMCSFPFVKNGHSESSGLIHLEGDTVQIICNTGYSLQNNEKNISCVERGWSTPPICSFTKGECHVPILEANVDAQPKKESYKVGDVLKFSCRKNLIRVGSDSVQCYQFGWSPNFPTCKGQVRSCGPPPQLSNGEVKEIRKEEYGHNEVVEYDCNPNFIINGPKKIQCVDGEWTTLPTCVEQVKTCGYIPELEYGYVQPSVPPYQHGVSVEVNCRNEYAMIGNNMITCINGIWTELPMCVATHQLKRCKIAGVNIKTLLKLSGKEFNHNSRIRYRCSDIFRYRHSVCINGKWNPEVDCTEKREQFCPPPPQIPNAQNMTTTVNYQDGEKVAVLCKENYLLPEAKEIVCKDGRWQSLPRCVESTAYCGPPPSINNGDTTSFPLSVYPPGSTVTYRCQSFYKLQGSVTVTCRNKQWSEPPRCLDPCVVSEENMNKNNIQLKWRNDGKLYAKTGDAVEFQCKFPHKAMISSPPFRAICQEGKFEYPICEAAAGGGSGLNDIFEAQKIEWHEGGGSHHHHHHHH

SEQ ID NO: 14 - protein, 483 aa, Mus musculus
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVSFTLAAAGGGSGLNDIFEAQKIEWHEGGGSHHHHHHHH

SEQ ID NO: 15 - DNA, 772 bp, Mus musculus
  misc_feature (1)..(772): cDNA Heavy Chain<CFHR1>M-5.1.5
tttttttttttttgctctgacagaggaggccggtcctggattcgattcccagttcctcacattcagtcagcactgaacacggacccctcaccatgaacttcgggctcagcttgattttccttgccctcattttaaaaggtgtccagtgtgaggtgcagctggtggagtctgggggagacttagtgaagcctggagggtccctgaaactctcctgtgcagcctctggattcactttcagtagctatgacatgtcttgggttcgccagactccagacaagaggctggagtgggtcgcaaccattagtagtggtggtagtaacacctactatccagacagtgtgaaggggcgattcaccatctccagagacaatgccaagaacaccctgtacctgcaaatgagcagtctgaagtctgaggacacagccatgtattactgtgcaagacaagggtattactacggtagtagttactatgctatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgc

SEQ ID NO: 16 - protein, 227 aa, Mus musculus
  MISC_FEATURE (1)..(227): Heavy Chain <CFHR1>M-5.1.5
MNFGLSLIFLALILKGVQCEVQLVESGGDLVKPGGSLKLSCAASGFTFSSYDMSWVRQTPDKRLEWVATISSGGSNTYYPDSVKGRFTISRDNAKNTLYLQMSSLKSEDTAMYYCARQGYYYGSSYYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVA

SEQ ID NO: 17 - DNA, 712 bp, Mus musculus
  misc_feature (1)..(712): cDNA coding for Light Chain <CFHR1>M-5.1.5
ggaccaaaattcaaagacaaaatggattctcaagtgcagattttcagcttccttctaatcagtgccttagtcataatgtccagaggacagattgttctcacccagtctccagcaatcatgtctgcatctccaggggagaaggtcaccatgacctgcagggccagttcaagtgtaagttccagttacttgcactggtaccagcagaagccaggatcttcccccaaactctggatttatatcacatccagcctggcttcaggagtcccagctcgcttcagtggcagtgggtctgggacctcttactctctcacaatcagcagtgtggaggctgaggatggtgccacttattactgccagcagtatcatagttccccgtatacgttcggatcggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaaga

SEQ ID NO: 18 - protein, 231 aa, Mus musculus
  MISC_FEATURE (1)..(231): Light Chain <CFHR1>M-5.1.5
MDSQVQIFSFLLISALVIMSRGQIVLTQSPAIMSASPGEKVTMTCRASSSVSSSYLHWYQQKPGSSPKLWIYITSSLASGVPARFSGSGSGTSYSLTISSVEAEDGATYYCQQYHSSPYTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKX

SEQ ID NO: 19 - protein, 8 aa, Mus musculus
  MISC_FEATURE (1)..(8): hCDR1 of 5.1.5
GFTFSSYD

SEQ ID NO: 20 - protein, 8 aa, Mus musculus
  MISC_FEATURE (1)..(8): hCDR2 of 5.1.5
ISSGGSNT

SEQ ID NO: 21 - protein, 16 aa, Mus musculus
  MISC_FEATURE (1)..(16): hCDR3 of 5.1.5
ARQGYYYGSSYYAMDY

SEQ ID NO: 22 - protein, 7 aa, Mus musculus
  MISC_FEATURE (1)..(7): LCDR1 of 5.1.5
SSVSSSY

SEQ ID NO: 23 - protein, 9 aa, Mus musculus
  MISC_FEATURE (1)..(9): LCDR3 of 5.1.5
QQYHSSPYT

SEQ ID NO: 24 - DNA, 631 bp, Mus musculus
  misc_feature (1)..(631): cDNA of the heavy chain of Mab 4.1.3
gaccacgcgtatcgatgtcgacttttttttttttttttncatcgctctcactggaggctgatctctgaagataaggaggtgtagcctaaaagatgagagtgctggttcttttgtggctgttcacagcctttcctggtatcctgtctgatgtgcagcttcaggagtcgggacctggcctggtgaaaccttctcagtctctgtccctcacctgcactgtcactggctactcaatcaccagtgaatatgcctggaactggatccggcaatttccaggaaacaaactggagtggatggcctacataaattccagtggtaaatctagctataatccatctctcaaaagtcgaatctctgtcactcgagacacatccaagaaccagttcttcctgcaattgaattctgtgactactgaggacacagccacatattactgtgcgagagagggatatggtaactacccggcctggtttgcttactggggccaagggactctggtcactgtctctgcagccaaaacaacagccccatcggtctatccactggcccctgtgtgtggagatacaactggctcctcggtgactctaggatgcctggtcaagggttatttccctgagccagtgaccttgacctg

SEQ ID NO: 25 - protein, 179 aa, Mus musculus
  MISC_FEATURE (1)..(179): heavy chain of Mab 4.1.3
MRVLVLLWLFTAFPGILSDVQLQESGPGLVKPSQSLSLTCTVTGYSITSEYAWNWIRQFPGNKLEWMAYINSSGKSSYNPSLKSRISVTRDTSKNQFFLQLNSVTTEDTATYYCAREGYGNYPAWFAYWGQGTLVTVSAAKTTAPSVYPLAPVCGDTTGSSVTLGCLVKGYFPEPVTLT

SEQ ID NO: 26 - DNA, 749 bp, Mus musculus
  misc_feature (1)..(749): cDNA sequence of the light chain of Mab 4.1.3
gaccacgagcacgcgtgtcgacttttttttttttttttgtawttgaagtcaagactcagcctggacatgatgtcctctgctcagttccttggtctcctgttgctctgttttcaaggtaccagatgtgatatccagatgacacagactacatcctccctgtctgcctctctgggagacagagtcaccatcagttgcagtgcaagtcaggacattaacaattatttaaactggtatcagcagaaaccagatggaactgttaaactcctgatttattacacttcaaatttacactcaggagtcccatcaaggttcagtggcagtgggtctgggacagattattctctcaccatcagcaacctggaacctgaagatattgccacttacttttgtcagcagtatagtaagcttcctcggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaactttcacccattgtcaaga

SEQ ID NO: 27 - protein, 227 aa, Mus musculus
  MISC_FEATURE (1)..(227): Light chain of Mab 4.1.3
MMSSAQFLGLLLLCFQGTRCDIQMTQTTSSLSASLGDRVTISCSASQDINNYLNWYQQKPDGTVKLLIYYTSNLHSGVPSRFSGSGSGTDYSLTISNLEPEDIATYFCQQYSKLPRTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTFTHCQ

SEQ ID NO: 28 - DNA, 599 bp, Mus musculus
  misc_feature (1)..(599): cDNA sequence of the heavy chain of Mab 4.2.53
gaccacgcgtatcgatgtcgacttttttttttttttttatctcctcactagagcccccatcagagcatggctgtcctggtgctgttcctctgcctggttgcatttccaagctgtgtcctgtcccaggtgcagttgaaggagtcaggacctggcctggtggcgccctcacagagcctgtccatcacttgcactgtctctgggttttcattaaccagttatggtgtacactgggttcgccagcctccaggaaagggtctggagtggctgggagtaatatgggctggtggaaatacagattatatttcggctctcatgtccagactgagcatcagcaaagacaactccaagagccaagttttcttaaaaataaacagactgcaaactgatgacacagccgtgtactactgtgccagagatcattatgatggttactacatggagtactggggtcaaggaacctcagtcaccgtctccccagccaaaacgacacccccatctgtctatccactggcccctggatctgctgcccaaactaactccatggtgaccctgggatgcctggtcaagggctatttccctgagccagtgacagtgacctg

SEQ ID NO: 29 - protein, 177 aa, Mus musculus
  MISC_FEATURE (1)..(177): heavy chain of Mab 4.2.53
MAVLVLFLCLVAFPSCVLSQVQLKESGPGLVAPSQSLSITCTVSGFSLTSYGVHWVRQPPGKGLEWLGVIWAGGNTDYISALMSRLSISKDNSKSQVFLKINRLQTDDTAVYYCARDHYDGYYMEYWGQGTSVTVSPAKTTPPSVYPLAPGSAAQTNSMVTLGCLVKGYFPEPVTVT

SEQ ID NO: 30 - DNA, 748 bp, Mus musculus
  misc_feature (1)..(748): cDNA sequence of the light chain of Mab 4.2.53
gaccacgcgtatcgatgtcgacttttttttttttttttagtcattcttggtcaggagacgttgtagaaatgagaccgtctattcagttcctggggctcttgttgttctggcttcatggtgttcagtgtgacatccagatgacacagtctccatcctcactgtctgcatctctgggaggcaaagtcaccatcacttgcaaggcaagccaggacattaacaaatatttagcttggtaccaacacaagcctggaaaaggtcctagactgctcatacattacacatttacattacagccaggcatcccatcaaggttcagtggaggtgggtctgggagagattattccttcaacatcaacaacctggagcctgaggatattgcaacttattattgtctgcagtatgataatctgtatacgttcgggggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaactttcacccattgtcaaga

SEQ ID NO: 31 - protein, 226 aa, Mus musculus
  MISC_FEATURE (1)..(226): Light chain of Mab 4.2.53
MRPSIQFLGLLLFWLHGVQCDIQMTQSPSSLSASLGGKVTITCKASQDINKYLAWYQHKPGKGPRLLIHYTFTLQPGIPSRFSGGGSGRDYSFNINNLEPEDIATYYCLQYDNLYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTFTHCQ

SEQ ID NO: 32 - DNA, 649 bp, Mus musculus
  misc_feature (1)..(649): cDNA sequence of the heavy chain of Mab 4.2.74
gaccacgcgtatcgatgtcgacttttttttttttttttwgctctgacagaggagccaagccctggattcccaggtcctcacattcagtgatcagcactgaacacagaccactcaccatggactccaggctcaatttagttttccttgtccttattttaaaaggtgtccagtgtgatgtgcggctggtggagtctgggggaggcttagtgcagcctggagggtcccggaaactctcctgtgcagcctctggattcactttcagtagctttggaatgcactgggttcgtcaggctccagagaaggggctggagtgggtcgcatacattagtagtggcagtagtaccatctactatgcagacacagtgaagggccgattcaccatctccagagacaatcccaagaacaccctgttcctgcaaatgaccagtctaaggtctgaggacacggccatgtattactgtgcaagatggggtaactcgtatgctatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctg

SEQ ID NO: 33 - protein, 177 aa, Mus musculus
  MISC_FEATURE (1)..(177): heavy chain of Mab 4.2.74
MDSRLNLVFLVLILKGVQCDVRLVESGGGLVQPGGSRKLSCAASGFTFSSFGMHWVRQAPEKGLEWVAYISSGSSTIYYADTVKGRFTISRDNPKNTLFLQMTSLRSEDTAMYYCARWGNSYAMDYWGQGTSVTVSSAKTTPPSVYPLAPGSAAQTNSMVTLGCLVKGYFPEPVTVT

SEQ ID NO: 34 - DNA, 744 bp, Mus musculus
  misc_feature (1)..(744): cDNA sequence of the light chain of Mab 4.2.74
ttttttttttttttttctacatctgaaaggcaggtggagcaagatggaatcacagactcaggtcctcatgtccctgctgttctgggtatctggtacctgtggggacattgtgatgacacagtctccatcctccctgactgtgacagcaggagagaaggtcactatgagctgcaagtccagtcagagtctgctaaacagaggaaatcaaaagaactatttgacctggtaccagcagaaaccagggcagcctcctaaactgttgatttactgggcatccactagggaatctggggtccctgatcgcttcacaggcagtggatctggaacagatttcactctcaccatcagcagtgtgctggctgaagacctggcagtttattactgtcagattgcttatagttctccattcacgttcggctcggggacaaagttggaaattaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaactttcacccattgtcaaga

SEQ ID NO: 35 - protein, 233 aa, Mus musculus
  MISC_FEATURE (1)..(233): light chain of Mab 4.2.74
MESQTQVLMSLLFWVSGTCGDIVMTQSPSSLTVTAGEKVTMSCKSSQSLLNRGNQKNYLTWYQQKPGQPPKLLIYWASTRESGVPDRFTGSGSGTDFTLTISSVLAEDLAVYYCQIAYSSPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTFTHCQ

SEQ ID NO: 36 - DNA, 653 bp, Mus musculus
  misc_feature (1)..(653): cDNA sequence of the heavy chain of Mab 5.3.23
gaccacgcgtatcgatgtcgacttttttttttttttttwgacagacgcacaaccctggactcccaagtctttctcttcagtgacaaacacagacatagaacattcaccatgtacttgggactgaacagtgtaatcatagtctttctcttaaaaggtgtccagagtgaagtgaagcttgaggagtctggaggaggcttggtgcaacctggaggatccatgaaactctcttgtgatggttctggattcacttttagtgatgcctggatgggctgggtccgccagtcaccagagaaggggcttgaatgggtttctgaaattagaaacagagctaataatcatgagacacattatgctgagtctgtgaaagggaggttcaccatctcaagagatgattccaaaagtagagtgtacttgcaaatgaacagtttaagagctgaagacactggcatttattactgtaacgggcggtattatgattcctactggttcctcgatgtctggggcgcagggaccacggtcaccgtctcctcagccaaaacgacacccccatctgtctatccactggcccctggatctgctgcccaaactaactccatggtgaccctgggatgcctggtcaagggctatttccctgagccagtgacagtgacctg

SEQ ID NO: 37 - protein, 181 aa, Mus musculus
  MISC_FEATURE (1)..(181): heavy chain of Mab 5.3.23
MYLGLNSVIIVFLLKGVQSEVKLEESGGGLVQPGGSMKLSCDGSGFTFSDAWMGWVRQSPEKGLEWVSEIRNRANNHETHYAESVKGRFTISRDDSKSRVYLQMNSLRAEDTGIYYCNGRYYDSYWFLDVWGAGTTVTVSSAKTTPPSVYPLAPGSAAQTNSMVTLGCLVKGYFPEPVTVT

SEQ ID NO: 38 - DNA, 729 bp, Mus musculus
  misc_feature (1)..(729): cDNA sequence of the light chain of Mab 5.3.23
tttttttttttttttttttgaaatacatcaggcaggcaaaggcatcaagatgaagtcacagacccaggtcttcatatttctactgctctgtgtgtctggtactcatgggaatattgtgatgacccagactcccaaattcctgcctgtgtcagtaggagacagggttaccatgacctgcaaggccagtcagagtgtgggttataatgtggcctggttccaacagaagccaggacagtctcctaaactgctgatatactatgcatccaatcgctacactggagtccctgatcgcttcactggcagtggaggtgggacagatttcactttcaccatcagcagtgtgcaggttgaagacctggcagtttatttctgtcagcagcattatatctctccaacgttcggatcggggaccaagcttgaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaactttcacccattgtcaaga

SEQ ID NO: 39 - protein, 226 aa, Mus musculus
  MISC_FEATURE: light chain of Mab 5.3.23
MKSQTQVFIFLLLCVSGTHGNIVMTQTPKFLPVSVGDRVTMTCKASQSVGYNVAWFQQKPGQSPKLLIYYASNRYTGVPDRFTGSGGGTDFTFTISSVQVEDLAVYFCQQHYISPTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTFTHCQ

SEQ ID NO: 40 - DNA, 616 bp, Mus musculus
  misc_feature (1)..(616): cDNA sequence of the heavy chain of Mab L20/3
gaccacgcgtatcgatgtcgacttttttttttttttttwacatatgtccaatgtcctctccacaggcactgaacacactgactctaaccatgggatggagctggatctttctcttcttcctgtcaggaactgcaggtgtcctctctgaggtcctgctgcaacagtctggacctgaactagtgaagcctggggcttcagtgaagataccctgcaaggcctctggatacacattcactgactacaacatggactgggtgaagcagagccatggaaagagccttgaatggattggagacattaatcctaacaatggttttactatctacaaccagaagttcaagggcaaggccacattgactgtagacaagtcctccagcacagcctacatggagctccgcagcctgacatctgaggacactgcaatctattactgtgcaatcaactgggactggtttgcttactggggccaagggactctggtcactgtctctgcagccaaaacgacacccccatctgtctatccactggcccctggatctgctgcccaaactaactccatggtgaccctgggatgcctggtcaagggctatttccctgagccagtgacagtgacctg

SEQ ID NO: 41 - protein, 175 aa, Mus musculus
  MISC_FEATURE (1)..(175): heavy chain of Mab L20/3
MGWSWIFLFFLSGTAGVLSEVLLQQSGPELVKPGASVKIPCKASGYTFTDYNMDWVKQSHGKSLEWIGDINPNNGFTIYNQKFKGKATLTVDKSSSTAYMELRSLTSEDTAIYYCAINWDWFAYWGQGTLVTVSAAKTTPPSVYPLAPGSAAQTNSMVTLGCLVKGYFPEPVTVT

SEQ ID NO: 42 - DNA, 778 bp, Mus musculus
  misc_feature (1)..(778): cDNA sequence of the light chain of Mab L20/3
gaccacgcgtagcgatgtcgacttttttttttttttttactgatcagtctcctcaggctgtctcctcaggttgcctcctcaaaatgaagttgcctgttaggctgttggtgctgatgttctggattcctgcttccagcagtgatgttttgatgacccaaactccactctccctgcctgtcagtcttggagatcaagcctccatctcttgcagatctagtcagagcattgtacataataatggaaacacctatttagaatggtacctgctgaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaactttcacccattgtcaaga

SEQ ID NO: 43 - protein, 231 aa, Mus musculus
  MISC_FEATURE (1)..(231): light chain of Mab L20/3
MKLPVRLLVLMFWIPASSSDVLMTQTPLSLPVSLGDQASISCRSSQSIVHNNGNTYLEWYLLKPGQSPKLLIYKVSNRFSGVPDRFSGSGSGTDFTLKISRVEAEDLGVYYCFQGSHVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTFTHCQ